(19) United States
(12) Patent Application Publication
(10) Pub. No.: US 2006/0031959 A1
(43) Pub. Date: Feb. 9, 2006

(54) Nucleic acid fragments encoding proteins involved in stress response

(76) Inventors: Saverio Carl Falco, (US); Omolayo O. Famodu, (US); Blake C. Meyers, (US); Guo-Hua Miao, (US); Joan T. Odell, (US); J. Antoni Rafalski, (US); Catherine J. Thorpe, (US); Hajime Sakai, (US); Zude Weng, (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/031,206

(22) Filed: Jan. 7, 2005

Related U.S. Application Data
(62) Division of application No. 10/078,929, filed on Feb. 19, 2002, now abandoned.
Application No. 10/078,929 is a division of application No. 09/566,394, filed on May 5, 2000, now abandoned.
(60) Provisional application No. 60/133,038, filed on May 7, 1999.
(60) Provisional application No. 60/133,042, filed on May 7, 1999.
(60) Provisional application No. 60/133,427, filed on May 11, 1999.
(60) Provisional application No. 60/133,437, filed on May 11, 1999.
(60) Provisional application No. 60/133,428, filed on May 11, 1999.
(60) Provisional application No. 60/133,438, filed on May 11, 1999.
(60) Provisional application No. 60/133,436, filed on May 11, 1999.
(60) Provisional application No. 60/137,667, filed on Jun. 4, 1999.

(51) Int. Cl. A01H 5/00; C07H 21/04; C12N 15/82; C12N 5/04

(52) U.S. Cl. 800/278; 435/412; 435/468; 530/370; 536/23.6; 800/320.1; 800/320.2; 800/320.3; 800/312

(57) ABSTRACT

The invention provides isolated peptide-methionine sulfoxide reductase nucleic acids and their encoded proteins. The present invention provides methods and compositions relating to altering peptide-methionine sulfoxide reductase levels in plants. The invention further provides recombinant expression cassettes, host cells, transgenic plants, and antibody compositions.

### RELATED APPLICATIONS

This application claims priority to U.S. Provisional Application Nos. 60/133,038, filed May 7, 1999; 60/133,042, filed May 7, 1999; 60/133,427 filed May 11, 1999; 60/133,437, filed May 11, 1999; 60/133,428, filed May 11, 1999; 60/133,438, filed May 11, 1999; 60/133,436, filed May 11, 1999; and 60/137,667, file Jun. 4, 1999, all of which are incorporated herein by reference.

### TECHNICAL FIELD

The present invention relates generally to plant molecular biology. More specifically, it relates to nucleic acids and methods for modulating their expression in plants.

### BACKGROUND OF THE INVENTION

Plants are constantly battered by stress in a variety of forms, which run the gamut from abiotic factors like drought, heat, and harmful radiation, to biotic factors like pathogen attack. Consequently, they have evolved an array of survival strategies to handle different stress conditions.

DNA Repair and Recombination Genes

Mutagens such as toxic chemicals and ionizing radiation may damage DNA. DNA repair is an integral cellular process that serves to minimize transmission of such DNA damage to daughter cells, thereby maintaining the integrity of the genetic material. Living cells have evolved a series of repair pathways appropriate for different types of DNA damage. These include photoreactivating enzymes, alkyltransferases, excision-repair, and postreplication repair.

A number of proteins involved in DNA repair have been identified and the corresponding genes cloned, including RAD26 from yeast (Guzder, S. N. et al., (1996) J. Biol. Chem. 271:18314-18317) and DRT111 and DRT112 from Arabidopsis (Pang, Q. et al., (1993) Nucl. Acids Res. 21:1647-1653). RAD26, in which null mutations severely reduce efficiency of transcription-coupled repair, encodes a DNA-dependent ATPase with no apparent DNA helicase activity. Meanwhile, DRT111 and DRT112 have been shown to increase resistance of E. coli ruvC recG mutants to UV light and several chemical DNA-damaging agents; the DRT111-encoded protein is not significantly homologous to any protein in the public database, whereas the DRT112-encoded protein is highly homologous to plastocyanin.

Isolating more genes involved in DNA repair may shed more light on that process and possibly on recombination, since both are closely related, as some DNA repair is accomplished via recombination. Recombination is the process by which DNA molecules are broken and rejoined, giving rise to new combinations. It is a key biological mechanism in mediating genetic diversity and DNA repair. Much research has focused on describing the process, since it is an integral biological phenomenon and as such, forms the basis of a number of practical applications ranging from molecular cloning to introduction of transgenes.

A number of proteins involved in recombination have been isolated and the corresponding genes cloned, including RecA of E. coli, a key player in the recombination process. RecA catalyzes the pairing up of a DNA double helix and a homologous region of single-stranded DNA, and so initiates the exchange of strands between two recombining DNA molecules. It exhibits DNA-dependent ATPase activity, binding DNA more tightly when it has ATP bound than when it has ADP bound. RecA gene homologues in other organisms have been isolated, including RAD51 from human, mouse and yeast (Shinohara, A. et al., (1993) Nat. Genet. 4:239-243), and DMC1 from yeast, lily, and Arabidopsis (Klimyuk, V. I. and Jones, J. D., (1997) Plant J. 11:1-14). In fission yeast, a number of meiotic recombination genes have been identified by genetic complementation, including rec6 and rec12 (Lin, Y. and Smith, G. R., (1994) Genetics 136:769-779).

Obtaining targeted knockouts of endogenous genes through introduction of homologous strands of DNA is a feat which has been achieved in mammalian cells several years ago. It is however an enormous challenge in plants, which is indicative of a lack of sufficient knowledge about homologous recombination in plant cells. Isolation and characterization of plant genes involved in recombination may help in overcoming the present obstacles.

Oxidative Stress Genes

Produced either as a defense response strategy or as byproducts of normal aerobic metabolism, active oxygen species which include superoxide radicals, hydrogen peroxide and hydroxyl radicals may cause oxidative damage to DNA, proteins, and lipids. This may lead to genetic lesions, and accelerated cellular aging and death. Cells have evolved a series of mechanisms to handle such oxidative stress, which include the production of enzymes such as superoxide dismutase (SOD) that catalase and detoxify the active oxygen species. Recently, msrA, which encodes a peptide-methionine sulfoxide reductase and ATX1, which encodes a small metal homeostasis factor have been found to provide resistance to oxidative stress (Lin, S. J., and Culotta, V. C., (1995) Proc. Natl. Acad. Sci. USA 92:3784-3788; Moskovitz J. et al., (1998) Proc. Natl. Acad. Sci. USA 95:14071-14075).

Peptide-methionine sulfoxide reductase is an enzyme that reduces protein methionine sulfoxide residues back to methionine. Its overexpression has been shown to enhance survival of yeast and human T lymphocytes under conditions of oxidative stress (Moskovitz J. et al., supra).

ATX1 was originally isolated by its ability to suppress oxygen toxicity in SOD-deficient yeast cells. The gene encodes a small polypeptide that is involved in the transport and/or partitioning of copper, a function that appears directly related to its ability to suppress oxygen toxicity. Yeast cells lacking a functional ATX1 gene were more sensitive to free radicals. ATX1 homologues have been identified in humans, called HAH1 (Klomp, L. W. et. al., (1997) J. Biol. Chem. 272:9221-9226), and Arabidopsis, called CCH (Himelblau E. et al., (1998) Plant Physiol. 117:1227-1234).

Isolation of more functional homologues of msrA and ATX1 in plants may potentially lead to a better understanding of how these genes are able to provide protection against oxidative stress, and identification of more genes and proteins involved in the process. Thus in the future, transgenic plants may be generated that overexpress these antioxidant molecules and are able to survive better under oxidative stress.

Additionally, the nucleic acid fragments of the instant invention may be used to create transgenic plants in which the disclosed peptide-methionine sulfoxide reductase or copper homeostasis factor is present at higher or lower levels than normal or in cell types or developmental stages in which they are not normally found. This would have the effect of altering the level of resistance to oxidative stress in those cells. Additionally, lower levels of oxidation resulting from overexpression of the disclosed peptide-methionine sulfoxide reductase or copper homeostasis factor may also protect flavor of grains such as rice.

Defense Response Genes

Plants synthesize signaling molecules in response to wounding, herbivore and pathogen attack. Phytoalexins are low molecular weight metabolites which plants accumulate in response to microbial infection. Phytoalexins accumulate at the site of bacterial and fungal infections at concentrations sufficient to inhibit development of the microbe eliciting a resistance response. This response may be brought forth by components of the host or the microbe cell wall or cell surfaces. Genes encoding carnation N-hydroxycinnamoyl-transferase have been described. These genes are constitutively expressed in cell cultures and are elicited in response to fungal infection (Yang, Q. et al. (1997) Plant Mol. Biol. 35:777:789). The product of these genes is also called anthranilate N-hydroxycinnamoyl/benzoyltransferases (HCBTs) and catalyzes the committed reaction in carnation phytoalexin biosynthesis. Analysis of the transcription and promoter sequences shows a conserved TATA box, three elicitor response elements and several other features involved in the elicitor regulation of HCBT (Yang, Q. et al. (1998) Plant Mol. Biol. 38:1201-1214).

Salicylic acid also induces defense responses in plants including kinases and glucosyltransferases. Tobacco genes induced immediately after salicylic acid or cyclohexamide treatment have been identified as UDP-glucose: flavonoid glucosyl transferases. These genes are also induced upon treatment with methyljasmonate, benzoic acid, acetylsalicylic acid, 2,4-dichlorophenoxyacetic acid and hydrogen peroxide but are not affected by other elicitors (Hovarth, D. M. and Chua, N. H. (1996) Plant Mol. Biol. 31:1061-1072). These tobacco genes are referred to as TOGTs and are also induced by fungal and avirulent pathogens. TOGT proteins expressed in E. coli show high glucosyltransferase activity towards hydroxycoumarins and hyrdoxycinnamic acids. TOGTs may function to conjugate aromatic metabolites prior to their transport and cross-linking to the cell wall (Fraissinet-Tachet, L. et al. (1998) FEBS Lett. 437:319-323).

Understanding of the genes involved in stress resistance in crop plants will allow the manipulation of these genes to create plants with broad disease resistance and stress tolerance.

Pathogenesis-Related Genes

Plants respond to bacterial, fungal and viral infections by accumulating a series of pathogen-related proteins (PR). Infection of the plant by avirulent pathogens causes rapid programmed cell death, called hypersensitive response. PR-1 proteins were first identified as being induced by the infection of tobacco by tobacco mosaic virus. The tobacco cDNAs encoding PR-1 were found to be of at least three different classes with each class containing many diverse members (Pfitzner, U. M. and Goodman, H. M. (1987) Nucleic Acids Res. 15:4449-4465). The wheat PR—I proteins are induced by fungal pathogens but not by salicylic acid or other systemic acquired resistance activators (Molina, A. et al. (1999) Mol. Plant Micorbe Interact. 12:53-58). All PR—I proteins have a signal sequence of some length and accumulate in the intercellular fluid. cDNAs encoding PR—I protein homologs have been identified in human, nematodes, tobacco, barley, wheat, tomato, rice and corn but many members are still to be identified. Identification of the genes encoding PR-1 homologs in all crops will help in understanding the plant defense mechanisms.

Disease Resistance Genes

A major step towards unraveling the molecular basis of pathogen race-specific resistance in plant-pathogen interactions has been the molecular isolation and characterization of plant disease resistance (R) genes whose encoded proteins recognize avirulence (avr) gene products of the pathogen. According to the gene-for-gene hypothesis (Staskawicz et al. (1995) Science 268:661-667), a particular plant-pathogen interaction would result in resistance if the host plant carried the R gene that corresponded to the avr gene present in the attacking pathogen race; otherwise, if either the R gene or the cognate avr gene or both were absent, a susceptible phenotype would be observed.

In the past few years, several plant R genes that confer resistance to a variety of viral, bacterial, and fungal pathogens have been cloned from different plant species. It is remarkable that despite their specificity, these R proteins share significant sequence similarity so that they can be grouped into classes based on the presence of particular protein domains. The class with the most members is the so-called NBS-LRR (for nucleotide-binding site, leucine-rich repeat) type of R proteins. As the name implies, member proteins have a nucleotide-binding site by the N-terminal region and irregular leucine-rich repeats towards the C-terminal region, with the length and the number of the repeats varying from member to member. Members include the Arabidopsis RPS2 gene which confers resistance to Pseudomonas syringae carrying the avirulence gene avrRpt2 (Mindrinos, M. et al., (1994) Cell 78:1089-1099; Bent, A. F. et al., (1994) Science 265:1856-1860), and the Arabidopsis RPM1 gene which confers resistance to Pseudomonas syringae carrying the avirulence genes avrRpm1 or avrB (Grant, M. R. et al., (1995) Science 269:843-846).

Isolation of more NBS-LRR R homologues will provide an array of potential disease resistance proteins from which R proteins with increased efficiency or novel pathogen specificities may be generated. These can then be introduced into crop plants, and along with other plant protection strategies, may comprise a multi-faceted approach to combating pathogens, thus offering disease resistance that will prove more durable over time.

Protein Transport Genes

Many of the proteins involved in stress response such as disease resistance genes (Song et al., (1995) Science 270:1804-1806), arabinogalactans (Majewska-Sawka and Nothnagel, (2000) Plant Physiol 122:3-9), glutathione S-transferase (Gronwald and Plaisance (1998) Plant Physiol 117:877-892), peroxidase (Christensen et al. (1998) Plant Physiol 118:125-135), and chitinase (Ancillo et al. (1999) Plant Mol Biol 39:1137-1151) are either transported to particular cellular compartments (like the plasma membrane or cell surface) or glycosylated or both, which mean that they undergo processing in the endoplasmic reticulum (ER) and the Golgi. Accordingly, a better understanding of the process involved in the protein transport mechanisms from the ER to the Golgi to the final destination of a particular protein may provide insights on how to streamline the plant response to stress. Additionally, manipulation of the levels of Golgi adaptor subunits in plants may produce larger amounts of coated vesicles allowing the plant to more efficiently detoxify itself by secretion.

Membrane-bound proteins, storage proteins and proteins destined for secretion are translated on the rough endoplasmic reticulum (ER) by membrane-bound ribosomes. These proteins will either remain in the ER membrane or, after proper folding, will travel through the Golgi apparatus towards their final destination. Transport through the Golgi is a stepwise process where the proteins are post-translationally modified (by the addition of sugars) before being deposited in their respective destinations. Proteins are transported to their final destinations in vehicles known as coated vesicles. This name is derived from the fact that the vesicles are coated by a protein (clathrin) which acts as a scaffold to promote vesicle formation. The vesicles bud from their membrane of origin and fuse at their destination preserving the orientation of the membrane structure. These vesicles transport materials from the Golgi to the vacuoles or plasma membrane and vice versa.

Adaptors are protein complexes which link clathrin to transmembrane receptors in the coated pits or vesicles. There are two clathrin-coated adaptor complexes in the cell one associated with the Trans-Golgi Network and one associated with the plasma membrane. The Golgi membrane adaptor complex (AP-1) contains at least four subunits: gamma-adaptin, beta′-adaptin, AP-47 and AP-19 while the plasma membrane adaptor complex (AP-2) contains alpha-adaptin, beta-adaptin, AP-50 and AP-17. The AP-2 adaptor complex is involved in the clathrin-mediated endocytosis of receptors.

Adaptins are essential for the formation of clathrin coated vesicles in the course of intracellular transport of receptor-ligand complexes. Gamma adaptin is composed of two domains separated by a hinge containing a proline and a glycine-rich region (Robinson, M. S. (1990) J. Cell Biol. 111:2319-2326). cDNAs encoding gamma-adaptin have been identified in mice, bovine, rat, human, yeasts, fungus and Arabidopsis, but no other plant gamma-adaptins have been identified to date. The smallest component of the Golgi adaptor is AP-19. cDNAs encoding AP-19 have been identified in rat, mouse, human, yeast, Arabidopsis and Camptotheca acuminata. In C. acuminata a small gene family expresses AP-19 throughout the plant (Maldonado-Mendoza, I. E. and Nessler, C. L. (1996) Plant Mol. Biol. 32:1149-1153).

Analysis of the amino acid sequence encoding the bovine beta-adaptin indicates that it contains two domains with the C-terminal domain being involved in receptor selection (Kirchhausen, T. et al. (1989) Proc. Natl. Acad. Sci. USA 86:2612-2616). Beta-adaptin cDNAs have been identified in rat, mouse, human, yeasts and bovine. Although no plant beta-adaptin sequences have been identified to date the clathrin-coated vesicles from zucchini contain a beta-type adaptin (Holstein, S. E. et al. J. (1994) Cell Sci. 107:945-953).

Identification, isolation and characterization of more nucleic acid fragments encoding adaptor complex subunits may lead to a better understanding of intracellular transport in general.

### SUMMARY OF THE INVENTION

Generally, it is the object of the present invention to provide nucleic acids and proteins relating to stress response, including but not limited to peptide methionine sulfoxide reductase. It is an object of the present invention to provide transgenic plants comprising the nucleic acids of the present invention, and methods for modulating expression of the nucleic acids of the present invention in a transgenic plant.

Therefore, in one aspect the present invention relates to an isolated nucleic acid comprising a member selected from the group consisting of (a) a polynucleotide having a specified sequence identity to a polynucleotide encoding a polypeptide of the present invention; (b) a polynucleotide which is complementary to the polynucleotide of (a); and, (c) a polynucleotide comprising a specified number of contiguous nucleotides from a polynucleotide of (a) or (b). The isolated nucleic acid can be DNA.

In other aspects the present invention relates to: 1) recombinant expression cassettes, comprising a nucleic acid of the present invention operably linked to a promoter, 2) a host cell into which has been introduced the recombinant expression cassette, and 3) a transgenic plant comprising the recombinant expression cassette. The host cell and plant are optionally from either maize, wheat, rice, or soybean.

### DETAILED DESCRIPTION OF THE INVENTION

Overview

A. Nucleic Acids and Protein of the Present Invention

Unless otherwise stated, the polynucleotide and polypeptide sequences identified in Table 1 represent polynucleotides and polypeptides of the present invention. Table 1 cross-references these polynucleotides and polypeptides to their gene name and internal database identification number. A nucleic acid of the present invention comprises a polynucleotide of the present invention. A protein of the present invention comprises a polypeptide of the present invention.

Table 1 lists the polypeptides that are described herein, the designation of the cDNA clones that comprise the nucleic acid fragments encoding polypeptides representing all or a substantial portion of these polypeptides, and the corresponding identifier (SEQ ID NO:) as used in the attached Sequence Listing. Table 1 also identifies the cDNA clones as individual ESTs (“EST”), the sequences of the entire cDNA inserts comprising the indicated cDNA clones (“FIS”), contigs assembled from two or more ESTs (“Contig”), contigs assembled from an FIS and one or more ESTs (“Contig*”), or sequences encoding the mature protein derived from an EST, FIS, a contig, or an FIS and PCR (“CGS”). Nucleotide SEQ ID NOs:1, 3, 7, 11, 13, 15, 19, 23, and 27 correspond to nucleotide SEQ ID NOs:1, 3, 5, 7, 17, 9, 11, 13, and 15, respectively, presented in U.S. Provisional Application No. 60/133,437, filed May 11, 1999. Amino acid SEQ ID NOs:2, 4, 8, 12, 14, 16, 20, 24, and 28 correspond to amino acid SEQ ID NOs:2, 4, 6, 8, 18, 10, 12, 14, and 16, respectively, presented in U.S. Provisional Application No. 60/133,437, filed May 11, 1999. Nucleotide SEQ ID NOs:31, 35, 39, and 43 correspond to nucleotide SEQ ID NOs:1, 3, 5, and 7, respectively, presented in U.S. Provisional Application No. 60/133,038, filed May 7, 1999. Amino acid SEQ ID NOs:32, 36, 40, and 44 correspond to amino acid SEQ ID NOs: 2, 4, 6, and 8, respectively, presented in U.S. Provisional Application No. 60/133,038, filed May 7, 1999. Nucleotide SEQ ID NO:47 corresponds to nucleotide SEQ ID NO: 7 presented in U.S. Provisional Application No. 60/133,438, filed May 11, 1999. Amino acid SEQ ID NO:48 corresponds to amino acid SEQ ID NO:8 presented in U.S. Provisional Application No. 60/133,438, filed May 11, 1999. Nucleotide SEQ ID NOs:49, 53, 57, 61, 67, 69, 73, and 77 correspond to nucleotide SEQ ID NOs:1, 3, 5, 7, 10, 12, 14, and 16, respectively, presented in U.S. Provisional Application No. 60/133,042, filed May 7, 1999. Amino acid SEQ ID NOs: 50, 54, 58, 62, 68, 70, 74, and 78 correspond to amino acid SEQ ID NOs:2, 4, 6, 8, 11, 13, 15, and 17, respectively, presented in U.S. Provisional Application No. 60/133,042, filed May 7, 1999. Nucleotide SEQ ID NOs:81, 83, 87, 91, 93, and 97 correspond to nucleotide SEQ ID NOs:1, 3, 5, 7, 9, and 11, respectively, presented in U.S. Provisional Application No. 60/133,427 filed May 11, 1999. Amino acid SEQ ID NOs:82, 84, 88, 92, 94, and 98 correspond to amino acid SEQ ID NOs:2, 4, 6, 8, 10, and 12, respectively, presented in U.S. Provisional Application No. 60/133,427 filed May 11, 1999. Nucleotide SEQ ID NOs:101, 103, 107, 111, 113, 117, 119, 123, 127, 129, 133, 135, and 139 correspond to nucleotide SEQ ID NOs:1, 3, 5, 7, 9, 11, 13, 15, 17, 19, 21, 23, and 25, respectively, presented in U.S. Provisional Application No. 60/137,667, filed Jun. 4, 1999. Amino acid SEQ ID NOs:102, 104,1108, 112, 114, 118, 120, 124, 128, 130, 134, 136, and 140 correspond to amino acid SEQ ID NOs:2, 4, 6, 8, 10, 12, 14, 16, 18, 20, 22, 24, and 26, respectively, presented in U.S. Provisional Application No. 60/137,667, filed Jun. 4, 1999. Nucleotide SEQ ID NOs:141, 145, 149, 153, 155, 157, and 161 correspond to nucleotide SEQ ID NOs:9, 11, 13, 15, 17, 19, 21, respectively, presented in U.S. Provisional Application No. 60/133,428, filed May 11, 1999. Amino acid SEQ ID NOs:142, 146, 150, 154, 156, 158, 162 correspond to amino acid SEQ ID NOs:10, 12, 14, 16, 18, 20, 22, respectively, presented in U.S. Provisional Application No. 60/133,428, filed May 11, 1999. Nucleotide SEQ ID NOs:165, 169, 173, and 177 correspond to nucleotide SEQ ID NOs:1, 3, 5, and 7, respectively, presented in U.S. Provisional Application No. 60/133,428, filed May 11, 1999. Amino acid SEQ ID NOs:166, 170, 174, and 178 correspond to amino acid SEQ ID NOs:2, 4, 6, and 8, respectively, presented in U.S. Provisional Application No. 60/133,428, filed May 11, 1999. The sequence descriptions and Sequence Listing attached hereto comply with the rules governing nucleotide and/or amino acid sequence disclosures in patent applications as set forth in 37 C.F.R. §1.821-1.825.

TABLE 1
Stress Response Proteins

| | | | SEQ ID NO: | |
|---|---|---|---|---|
| Protein (Plant Source) | Clone Designation | Status | (Polynucleotide) | (Polypeptide) |
| Peptide-methionine Sulfoxide | p0050.cj1aa26r | EST | 1 | 2 |
| Reductase (Corn) | | | | |
| Peptide-methionine Sulfoxide | rr1.pk079.o8 | EST | 3 | 4 |
| Reductase (Rice) | | | | |
| Peptide-methionine Sulfoxide | rr1.pk079.o8 | FIS | 5 | 6 |
| Reductase (Rice) | | | | |
| Peptide-methionine Sulfoxide | Contig of: | Contig | 7 | 8 |
| Reductase (Soybean) | sdp2c.pk009.k16 | | | |
| | sl2.pk0004.h5 | | | |
| Peptide-methionine Sulfoxide | sdp2c.pk009.k16 | CGS | 9 | 10 |
| Reductase (Soybean) | (FIS) | | | |
| Peptide methionine sulfoxide | wlm96.pk046.n12 | EST | 11 | 12 |
| Reductase (Wheat) | | | | |
| Copper Homeostasis Factor | chp2.pk0001.b11 | EST | 13 | 14 |
| (Corn) | | | | |
| Copper Homeostasis Factor | cr1.pk0032.a11 | EST | 15 | 16 |
| (Corn) | | | | |
| Copper Homeostasis Factor | cr1.pk0032.a11 | FIS | 17 | 18 |
| (Corn) | | | | |
| Copper Homeostasis Factor | res1c.pk007.h24 | EST | 19 | 20 |
| (Rice) | | | | |
| Copper Homeostasis Factor | res1c.pk007.h24 | CGS | 21 | 22 |
| (Rice) | (FIS) | | | |
| Copper Homeostasis Factor | sls1c.pk024.m18 | EST | 23 | 24 |
| (Soybean) | | | | |
| Copper Homeostasis Factor | sls1c.pk024.m18 | CGS | 25 | 26 |
| (Soybean) | (FIS) | | | |
| Copper Homeostasis Factor | wre1n.pk0042.e2 | EST | 27 | 28 |
| (Wheat) | | | | |
| Copper Homeostasis Factor | wre1n.pk0042.e2 | CGS | 29 | 30 |
| (Wheat) | (FIS) | | | |
| DRT111 Homolog (Corn) | Contig of: | Contig | 31 | 32 |
| | p0062.cymaj36r | | | |
| | p0113.cieab53r | | | |
| DRT111 Homolog (Corn) | p0062.cymaj36r | CGS | 33 | 34 |
| | (FIS) | | | |
| DRT111 Homolog (Soybean) | Contig of: | Contig | 35 | 36 |
| | sdp4c.pk0001.o13 | | | |
| | sgs5c.pk0003.e10 | | | |
| DRT111 Homolog (Soybean) | sdp4c.pk001.o13 | FIS | 37 | 38 |
| DRT111 Homolog (Wheat) | wr1.pk0018.g3 | EST | 39 | 40 |
| DRT111 Homolog (Wheat) | wr1.pk0018.g3 | FIS | 41 | 42 |
| RAD26 Homolog (Corn) | ctn1c.pk001.i10 | EST | 43 | 44 |
| RAD26 Homolog (Corn) | ctn1c.pk001.i10 | FIS | 45 | 46 |
| REC12 Homolog (Soybean) | sgs2c.pk003.h9 | EST | 47 | 48 |
| HCBT (Corn) | cr1n.pk0177.d10 | EST | 49 | 50 |
| HCBT (Corn) | cr1n.pk0177.d10 | CGS | 51 | 52 |
| | (FIS) | | | |
| HCBT (Rice) | Contig of: | Contig | 53 | 54 |
| | rlr2.pk0020.g3 | | | |
| | rlr48.pk0007.c9 | | | |
| HCBT (Rice) | rlr48.pk0007.c9 | CGS | 55 | 56 |
| | (FIS) | | | |
| HCBT (Soybean) | Contig of: | Contig | 57 | 58 |
| | sfl1.pk128.f13 | | | |
| | sfl1.pk126.j22 | | | |
| | src3c.pk022.p10 | | | |
| | ssm.pk0011.h11 | | | |
| HCBT (Soybean) | sfl1.pk126.j22 (FIS) | CGS | 59 | 60 |
| HCBT (Wheat) | wlmk8.pk0021.e3 | EST | 61 | 62 |
| HCBT (Wheat) | wlmk8.pk0021.e3 | CGS | 63 | 64 |
| | (FIS) | | | |
| Glucosyltransferase (Corn) | cpc1c.pk004.o20 | FIS | 65 | 66 |
| Glucosyltransferase (Corn) | p0084.clopa50r | EST | 67 | 68 |
| Glucosyltransferase (Rice) | rls6.pk0084.f4 | EST | 69 | 70 |
| Glucosyltransferase (Rice) | rls6.pk0084.f4 (FIS) | CGS | 71 | 72 |
| Glucosyltransferase (Soybean) | src3c.pk020.h17 | EST | 73 | 74 |
| Glucosyltransferase (Soybean) | src3c.pk020.h17 | CGS | 75 | 76 |
| | (FIS) | | | |
| Glucosyltransferase (Wheat) | wlm96.pk028.k4 | EST | 77 | 78 |
| Glucosyltransferase (Wheat) | wlm96.pk028.k4 | CGS | 79 | 80 |
| | (FIS) | | | |
| PR-1 (Corn) | p0037.crwaw93rb | EST | 81 | 82 |
| PR-1 (Rice) | rr1.pk077.e22 | EST | 83 | 84 |
| PR-1 (Rice) | rr1.pk077.e22 (FIS) | CGS | 85 | 86 |
| PR-1 (Soybean) | sdp4c.pk009.g7 | EST | 87 | 88 |
| PR-1 (Soybean) | sdp4c.pk009.g7 (FIS) | CGS | 89 | 90 |
| PR-1 (Soybean) | sls1c.pk010.p21 | EST | 91 | 92 |
| PR-1 (Soybean) | Contig of: | Contig | 93 | 94 |
| | src2c.pk023.b14 | | | |
| | srn1c.pk002.c19 | | | |
| PR-1 (Soybean) | src2c.pk023.b14 | CGS | 95 | 96 |
| | (FIS) | | | |
| PR-1 (Soybean) | srn1c.pk002.c19 | FIS | 207 | 208 |
| PR-1 (Wheat) | wlm96.pk025.j5 | EST | 97 | 98 |
| PR-1 (Wheat) | wlm96.pk025.j5 | CGS | 99 | 100 |
| | (FIS) | | | |
| NBS-LRR R protein (Corn) | Contig of: | Contig | 101 | 102 |
| | p0010.cbpbx77r | | | |
| | p0010.cbpbx77rx | | | |
| NBS-LRR R protein (Corn) | p0034.cdnad43r | EST | 103 | 104 |
| NBS-LRR R protein (Corn) | p0034.cdnad43r | FIS | 105 | 106 |
| NBS-LRR R protein (Corn) | p0130.cwtab66r | EST | 107 | 108 |
| NBS-LRR R protein (Corn) | p0130.cwtab66r | FIS | 109 | 110 |
| NBS-LRR R protein (Rice) | rca1c.pk005.116 | EST | 111 | 112 |
| NBS-LRR R protein (Rice) | rlr6.pk0059.e10 | EST | 113 | 114 |
| NBS-LRR R protein (Rice) | rlr6.pk0059.e10 | FIS | 115 | 116 |
| NBS-LRR R protein (Rice) | rls6.pk0002.d12 | EST | 117 | 118 |
| NBS-LRR R protein (Soybean) | se4.pk0018.e4 | EST | 119 | 120 |
| NBS-LRR R protein (Soybean) | se4.pk0018.e4 | FIS | 121 | 122 |
| NBS-LRR R protein (Soybean) | sr1.pk0076.b9 | EST | 123 | 124 |
| NBS-LRR R protein (Soybean) | sr1.pk0076.b9 | FIS | 125 | 126 |
| NBS-LRR R protein (Soybean) | Contig of: | Contig | 127 | 128 |
| | sdp3c.pk016.l15 | | | |
| | src2c.pk004.f18 | | | |
| NBS-LRR R protein (Soybean) | src2c.pk028.d15 | EST | 129 | 130 |
| NBS-LRR R protein (Soybean) | src2c.pk028.d15 | FIS | 131 | 132 |
| NBS-LRR R protein (Wheat) | wlm0.pk0014.a2 | EST | 133 | 134 |
| NBS-LRR R protein (Wheat) | wlmk1.pk0020.h8 | EST | 135 | 136 |
| NBS-LRR R protein (Wheat) | wlmk1.pk0020.h8 | FIS | 137 | 138 |
| NBS-LRR R protein (Wheat) | wlmk8.pk0022.c11 | EST | 139 | 140 |
| AP-19 (Corn) | p0038.crvak82r | EST | 141 | 142 |
| AP-19 (Corn) | p0038.crvak82r (FIS) | CGS | 143 | 144 |
| AP-19 (Soybean) | srr1c.pk002.p3 | EST | 145 | 146 |
| AP-19 (Soybean) | srr1c.pk002.p3 (FIS) | CGS | 147 | 148 |
| AP-19 (Wheat) | wr1.pk148.a5 | EST | 149 | 150 |
| AP-19 (Wheat) | wr1.pk148.a5 (FIS) | CGS | 151 | 152 |
| AP-47 (Corn) | p0010.cbpcq26r | EST | 153 | 154 |
| AP-47 (Rice) | rr1.pk0024.g1 | EST | 155 | 156 |
| AP-47 (Soybean) | srr1c.pk003.g1 | EST | 157 | 158 |
| AP-47 (Soybean) | srr1c.pk003.g1 (FIS) | CGS | 159 | 160 |
| AP-47 (Wheat) | wre1n.pk0123.c3 | EST | 161 | 162 |
| AP-47 (Wheat) | wre1n.pk0123.c3 | FIS | 163 | 164 |
| Beta-adaptin (Corn) | p0119.cmtnr87r | EST | 165 | 166 |
| Beta-adaptin (Corn) | p0119.cmtnr87r | CGS | 167 | 168 |
| | (FIS) | | | |
| Beta-adaptin (Rice) | rls72.pk0017.g8 | EST | 169 | 170 |
| Beta-adaptin (Rice) | rls72.pk0017.g8 | FIS | 171 | 172 |
| Beta-adaptin (Soybean) | sml1c.pk004.n9 | EST | 173 | 174 |
| Beta-adaptin (Soybean) | sml1c.pk004.n9 | FIS | 175 | 176 |
| Beta-adaptin (Wheat) | wlm96.pk0001.b7 | EST | 177 | 178 |
| Beta-adaptin (Wheat) | wlm96.pk0001.b7 | FIS | 179 | 180 |
| Gamma-adaptin (Corn) | p0119.cmtoc10r | EST | 181 | 182 |
| Gamma-adaptin (Corn) | p0119.cmtoc10r | FIS | 183 | 184 |
| Gamma-adaptin (Rice) | rlr24.pk0087.a2 | EST | 185 | 186 |
| Gamma-adaptin (Rice) | rlr24.pk0087.a2 | CGS | 187 | 188 |
| | (FIS) | | | |
| Gamma-adaptin (Soybean) | sgs4c.pk001.j2 | EST | 189 | 190 |
| Gamma-adaptin (Soybean) | sgs4c.pk001.j2 (FIS) | CGS | 191 | 192 |
| Gamma-adaptin (Wheat) | wl1n.pk0038.d10 | EST | 193 | 194 |
| Gamma-adaptin (Wheat) | wl1n.pk0038.d10 | FIS | 195 | 196 |

cDNA clones encoding stress response proteins were identified by conducting BLAST (Basic Local Alignment Search Tool; Altschul et al. (1993) J. Mol. Biol. 215:403-410; see also www.ncbi.nlm.nih.gov/BLAST/) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences obtained in Example 1 were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish and States (1993) Nat. Genet. 3:266-272) provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

The BLASTX search using the sequences from clones listed in Table 1 revealed similarity of the polypeptides encoded by the cDNAs to various stress response proteins. Shown in Table 2 are the BLAST results for sequences enumerated in Table 1.

TABLE 2
BLAST Results for Sequences Encoding Polypeptides
Homologous to Stress Response Proteins

| | | Homologue NCBI | |
|---|---|---|---|
| | | GenBank | |
| SEQ ID | | Identifier (GI) | BLAST |
| NO: | Homologue Species | No. | pLog value |
| 2 | Lycopersicon esculentum | 1709692 | 51.15 |
| 4 | Arabidopsis thaliana | 4455256 | 58.70 |
| 6 | Lactuca sativa | 6635341 | 91.30 |
| 8 | Arabidopsis thaliana | 4455256 | 77.52 |
| 10 | Lactuca sativa | 6635341 | 96.30 |
| 12 | Arabidopsis thaliana | 4455256 | 45.30 |
| 14 | Arabidopsis thaliana | 3168840 | 26.40 |
| 16 | Saccharomyces cerevisiae | 584821 | 15.52 |
| 18 | Glycine max | 6525011 | 9.00 |
| 20 | Arabidopsis thaliana | 3168840 | 30.52 |
| 22 | Oryza sativa | 6525009 | 68.09 |
| 24 | Saccharomyces cerevisiae | 584821 | 7.00 |
| 26 | Arabidopsis thaliana | 3168840 | 6.70 |
| 28 | Arabidopsis thaliana | 3168840 | 25.52 |
| 30 | Oryza sativa | 6525009 | 37.15 |
| 32 | Arabidopsis thaliana | 1169200 | 22.70 |
| 34 | Arabidopsis thaliana | 1169200 | 120.00 |
| 36 | Arabidopsis thaliana | 1169200 | 67.30 |
| 38 | Arabidopsis thaliana | 1169200 | 84.70 |
| 40 | Arabidopsis thaliana | 1169200 | 66.30 |
| 42 | Arabidopsis thaliana | 1169200 | 62.00 |
| 44 | Saccharomyces cerevisiae | 550429 | 10.52 |
| 46 | Homo sapiens | 4557565 | 61.00 |
| 48 | Schizosaccharomyces pombe | 3123261 | 12.15 |
| 50 | Dianthus caryophyllus | 2239083 | 13.30 |
| 52 | Ipomoea batatas | 6469032 | 137.00 |
| 54 | Dianthus caryophyllus | 2239085 | 11.70 |
| 56 | Ipomoea batatas | 6469032 | 47.30 |
| 58 | Dianthus caryophyllus | 2239083 | 41.15 |
| 60 | Ipomoea batatas | 6469032 | 153.00 |
| 62 | Dianthus caryophyllus | 2239083 | 23.70 |
| 64 | Ipomoea batatas | 6469032 | 79.70 |
| 66 | Vigna mungo | 4115534 | 45.70 |
| 68 | Nicotiana tabacum | 1685005 | 32.52 |
| 70 | Nicotiana tabacum | 1685003 | 14.22 |
| 72 | Nicotiana tabacum | 1685005 | 81.70 |
| 74 | Nicotiana tabacum | 1685005 | 47.00 |
| 76 | Nicotiana tabacum | 1685005 | 144.00 |
| 78 | Nicotiana tabacum | 1685003 | 22.00 |
| 80 | Nicotiana tabacum | 1685005 | 103.00 |
| 82 | Triticum aestivum | 3702665 | 75.05 |
| 84 | Hordeum vulgare | 1076732 | 65.22 |
| 86 | Hordeum vulgare | 1076732 | 74.70 |
| 88 | Medicago truncatula | 2500715 | 26.40 |
| 90 | Medicago truncatula | 2500715 | 43.70 |
| 92 | Brassica napus | 1498731 | 51.70 |
| 94 | Nicotiana tabacum | 130846 | 56.00 |
| 96 | Nicotiana tabacum | 130846 | 47.70 |
| 98 | Zea mays | 3290004 | 57.05 |
| 100 | Zea mays | 3290004 | 64.70 |
| 102 | Avena sativa | 3411227 | >250 |
| 104 | Arabidopsis thaliana | 625973 | 29.70 |
| 106 | Arabidopsis thaliana | 625973 | 76.70 |
| 108 | Brassica napus | 4092774 | 34.00 |
| 110 | Oryza sativa | 4519938 | >254.00 |
| 112 | Arabidopsis thaliana | 625973 | 5.00 |
| 114 | Brassica napus | 4092771 | 13.70 |
| 116 | Oryza sativa | 4519938 | >254.00 |
| 118 | Hordeum vulgare | 2792210 | 27.00 |
| 120 | Brassica napus | 4092774 | 16.52 |
| 122 | Brassica napus | 4092771 | 26.15 |
| 124 | Oryza sativa | 4521190 | 17.05 |
| 126 | Brassica napus | 4092774 | 67.70 |
| 128 | Lycopersicon esculentum | 1513144 | 39.00 |
| 130 | Brassica napus | 4092771 | 10.52 |
| 132 | Arabidopsis lyrata | 5231014 | 12.40 |
| 134 | Hordeum vulgare | 2792212 | 38.30 |
| 136 | Brassica napus | 4092774 | 15.52 |
| 138 | Sorghum bicolor | 4680207 | 80.00 |
| 140 | Brassica napus | 4092771 | 22.50 |
| 142 | Camptotheca acuminata | 1762309 | 80.70 |
| 144 | Camptotheca acuminata | 1762309 | 82.00 |
| 146 | Arabidopsis thaliana | 2231702 | 75.00 |
| 148 | Camptotheca acuminata | 1762309 | 79.52 |
| 150 | Camptotheca acuminata | 1762309 | 65.52 |
| 152 | Camptotheca acuminata | 1762309 | 81.52 |
| 154 | Caenorhabditis elegans | 543816 | 59.70 |
| 156 | Mus musculus | 543817 | 39.00 |
| 158 | Mus musculus | 543817 | 27.22 |
| 160 | Mus musculus | 6671557 | 155.00 |
| 162 | Caenorhabditis elegans | 543816 | 43.30 |
| 164 | Drosophila melanogaster | 6492272 | 143.00 |
| 166 | Homo sapiens | 1703167 | 83.10 |
| 168 | Drosophila melanogaster | 481762 | >254.00 |
| 170 | Homo sapiens | 1703167 | 21.30 |
| 172 | Drosophila melanogaster | 481762 | 76.04 |
| 174 | Homo sapiens | 1703167 | 33.00 |
| 176 | Rattus norvegicus | 1703168 | 27.70 |
| 178 | Rattus norvegicus | 203115 | 25.30 |
| 180 | Drosophila melanogaster | 481762 | >254.00 |
| 182 | Arabidopsis thaliana | 3372671 | 52.52 |
| 184 | Arabidopsis thaliana | 4538987 | >254.00 |
| 186 | Arabidopsis thaliana | 3372671 | 52.00 |
| 188 | Arabidopsis thaliana | 4538987 | >254.00 |
| 190 | Arabidopsis thaliana | 3372671 | 36.00 |
| 192 | Arabidopsis thaliana | 4538987 | >254.00 |
| 194 | Arabidopsis thaliana | 3372671 | 34.22 |
| 196 | Arabidopsis thaliana | 4704741 | 94.00 |
| 208 | Nicotiana tabacum | 130846 | 34.0 |

NCBI GenBank Identifier (GI) Nos. 1709692, 4455256, and 6635341 are amino acid sequences of peptide-methionine sulfoxide reductase; NCBI GI Nos. 3168840, 584821, 6525011, and 6525009 are amino acid sequences of copper homeostasis factor; NCBI GI No. 1169200 is DRT111 amino acid sequence; NCBI GI No. 550429 is RAD26 amino acid sequence; NCBI GI No. 4557565 is RAD26 homolog amino acid sequence; NCBI GI No. 3123261 is REC12 recombination protein amino acid sequence; NCBI GI Nos. 2239083, 6469032, and 2239085 are HCBT amino acid sequences; NCBI GI Nos. 4115534, 1685005, and 1685003 are glucosyltransferase amino acid sequences; NCBI GI Nos. 3702665, 1076732, 2500715, 1498731, 130846, and 3290004 are pathogenesis-related (PR) protein amino acid sequences; NCBI GI Nos. 3411227, 625973, 4092774, 4519938, 4092771, 2792210, 4521190, 1513144, 5231014, 2792212, and 4680207 are NBS-LRR R protein amino acid sequences; NCBI GI Nos. 1762309 and 2231702 are AP19 amino acid sequences; NCBI GI Nos. 543816, 543817, 6671557, and 6492272 are AP47 amino acid sequences; NCBI GI Nos. 1703167, 481762, 1703168, 203115, and 481762 are beta-adaptin amino acid sequences; and NCBI GenBank Identifier GI Nos. 3372671, 4538987, and 4704741 are gamma-adaptin amino acid sequences.

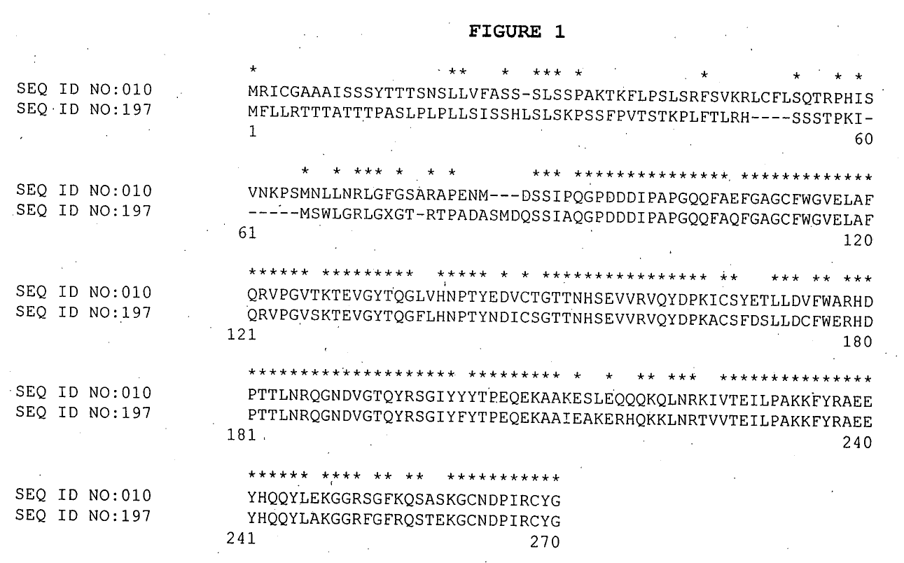

FIG. 1 depicts the amino acid sequence alignment between the peptide-methionine sulfoxide reductase encoded by the nucleotide sequence derived from soybean clone sdp2c.pk009.k16 (SEQ ID NO:10) and the Lactuca sativa peptide-methionine sulfoxide reductase (NCBI GenBank Identifier (GI) No. 6635341; SEQ ID NO: 197). Amino acids which are conserved between the two sequences are indicated with an asterisk (*). Dashes are used by the program to maximize alignment of the sequences. There is 65% identity between SEQ ID NOs:10 and 197.

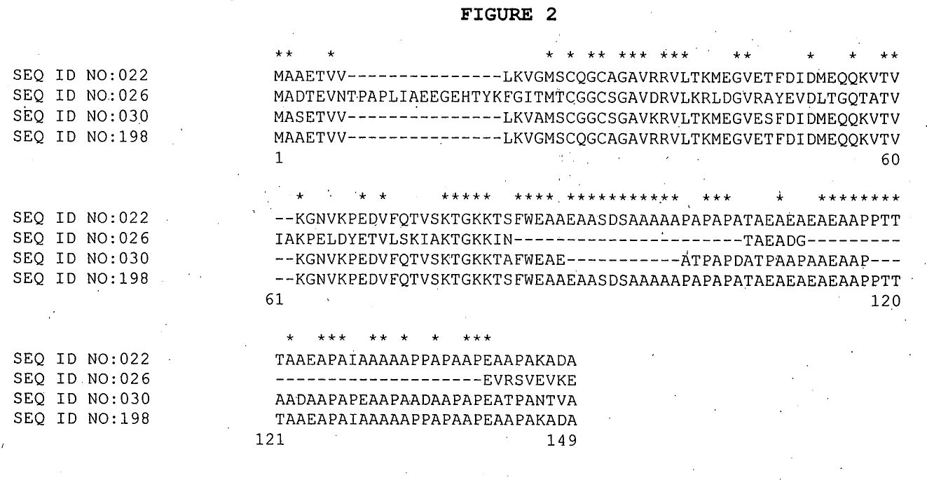

FIG. 2 depicts the amino acid sequence alignment between the copper homeostasis factor encoded by the nucleotide sequences derived from rice clone res1c.pk007.h24 (SEQ ID NO:22), soybean clone slslc.pk024.m18 (SEQ ID NO:26), and wheat clone wreln.pk0042.e2 (SEQ ID NO:30), and the copper homeostasis factor from rice (NCBI GenBank Identifier (GI) No. 6525009; SEQ ID NO:198). Amino acids which are conserved among all and at least two sequences with an amino acid at that position are indicated with an asterisk (*). Dashes are used by the program to maximize alignment of the sequences. There is 100% identity between SEQ ID NOs:22 and 198, 24% identity between SEQ ID NOs:26 and 198, and 69% identity between SEQ ID NOs:30 and 198.

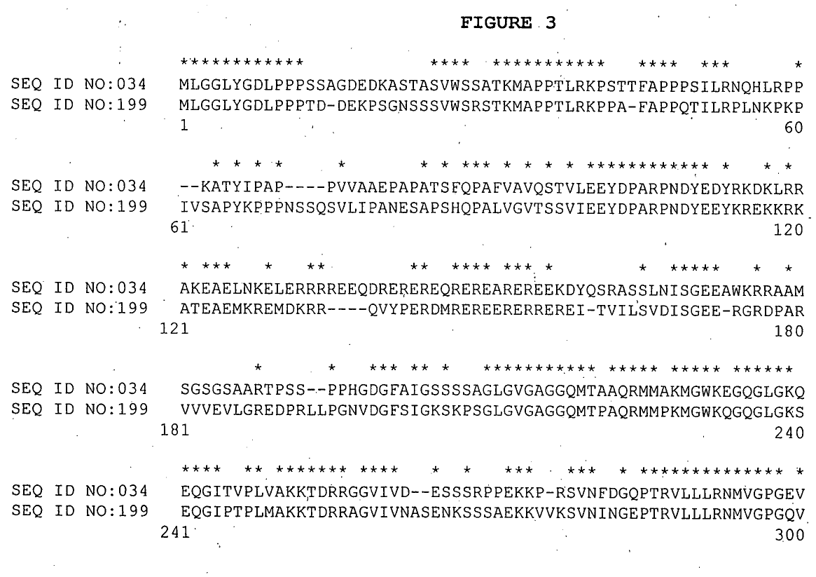

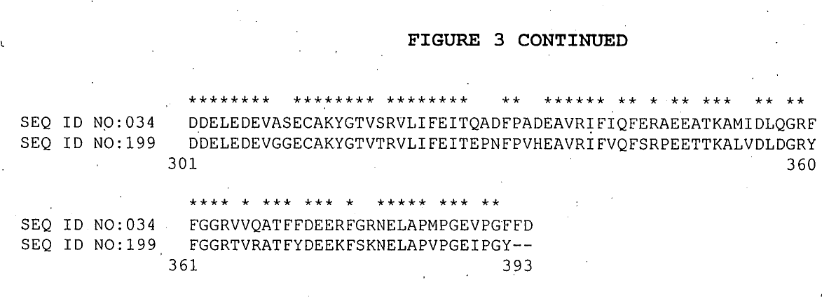

FIG. 3 depicts the amino acid sequence alignment between the DRT111 homolog encoded by the nucleotide sequence derived from corn clone p0062.cymaj36r (SEQ ID NO:34) and the DRT111 protein from Arabidopsis thaliana (NCBI GenBank Identifier (GI) No. 1169200; SEQ ID NO:199). Amino acids which are conserved between the two sequences are indicated with an asterisk (*). Dashes are used by the program to maximize alignment of the sequences. There is 54% identity between SEQ ID NOs:34 and 199.

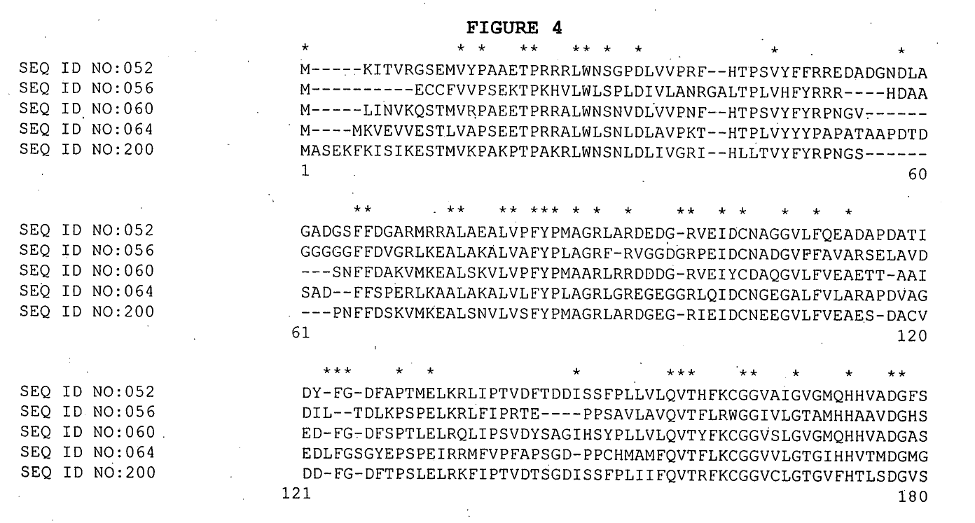

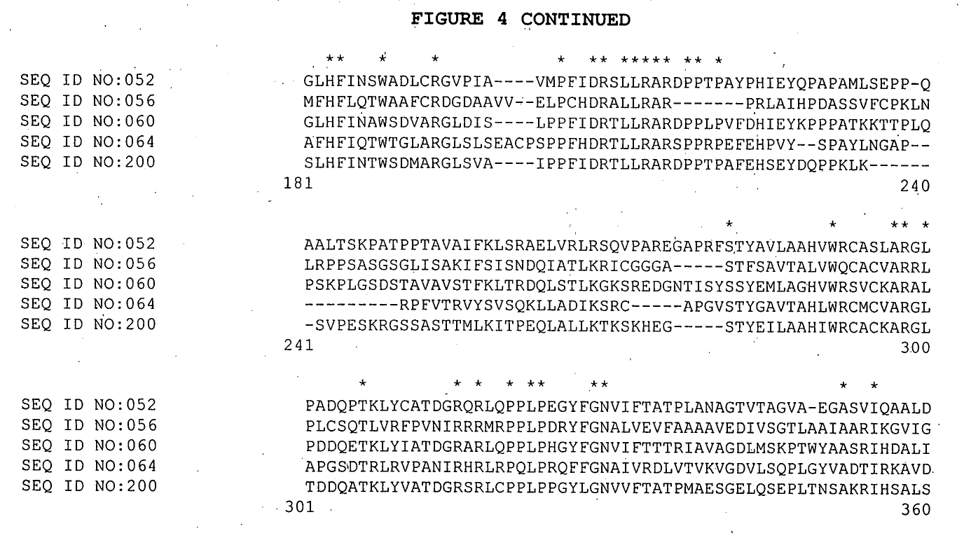

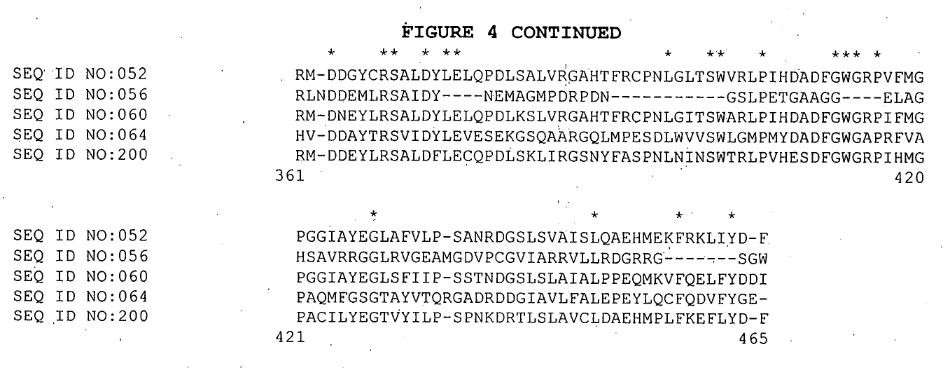

FIG. 4 depicts the amino acid sequence alignment between the HCBT encoded by the nucleotide sequences derived from corn clone crln.pk0177.d10 (SEQ ID NO:52), rice clone rlr48.pk0007.c9 (SEQ ID NO:56), soybean clone sfl1.pk126j22 (SEQ ID NO:60), and wheat clone wlmk8.pk0021.e3 (SEQ ID NO:64), and the HCBT from Ipomoea batatas (NCBI GenBank Identifier (GI) No. 6469032; SEQ ID NO:200). Amino acids which are conserved among all and at least two sequences with an amino acid at that position are indicated with an asterisk (*). Dashes are used by the program to maximize alignment of the sequences. There is 52% identity between SEQ ID NOs:52 and 200, 27% identity between SEQ ID NOs:56 and 200, 58% identity between SEQ ID NOs:60 and 200, and 33% identity between SEQ ID NOs:64 and 200.

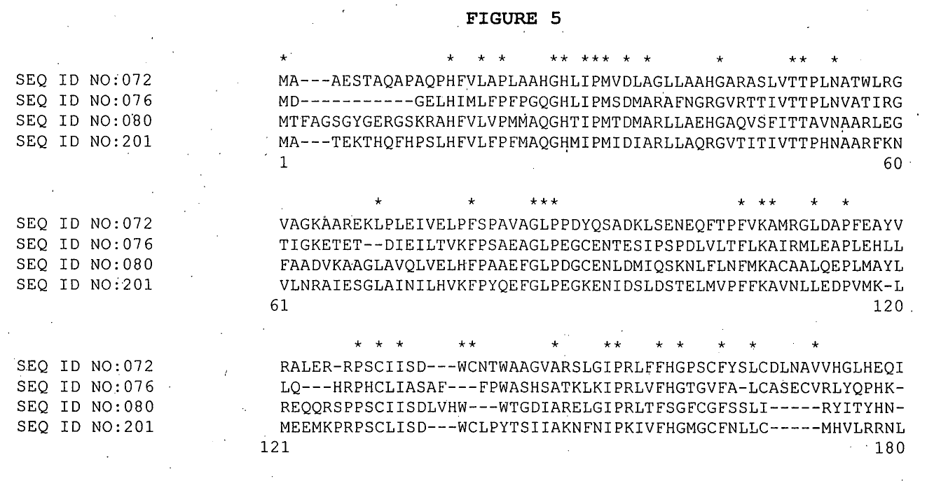

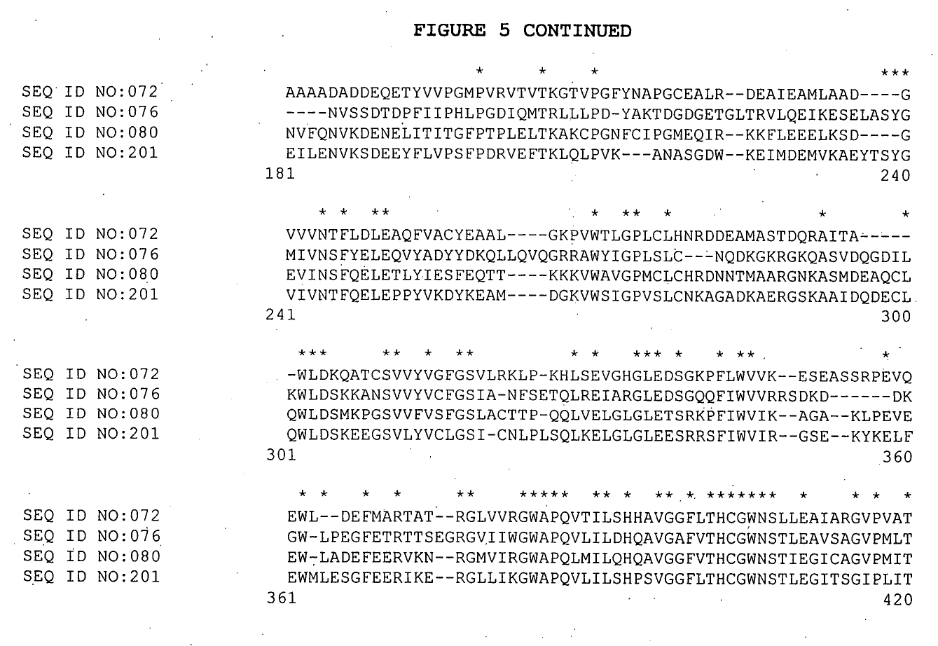

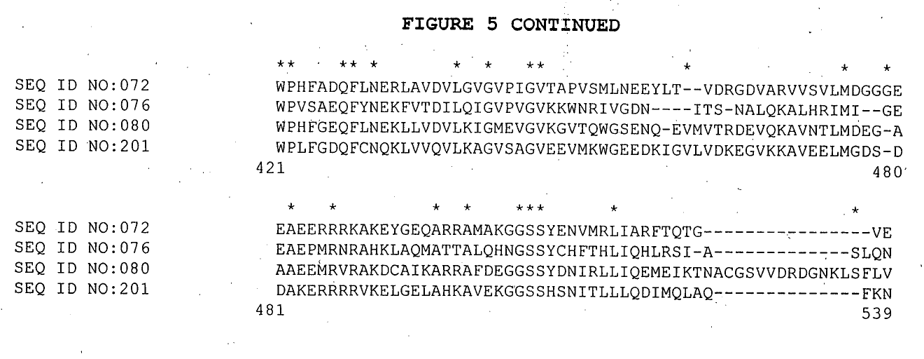

FIG. 5 depicts the amino acid sequence alignment between the glucosyltransferase encoded by the nucleotide sequences derived from rice clone rls6.pk0084.f4 (SEQ ID NO:72), soybean clone src3c.pk020.h17 (SEQ ID NO:76), and wheat clone wlm96.pk028.k4 (SEQ ID NO:80), and the glucosyltransferase from Nicotiana tabacum (NCBI GenBank Identifier (GI) No. 1685005; SEQ ID NO:201). Amino acids which are conserved among all and at least two sequences with an amino acid at that position are indicated with an asterisk (*). Dashes are used by the program to maximize alignment of the sequences. There is 37% identity between SEQ ID NOs:72 and 201, 37% identity between SEQ ID NOs:76 and 201, and 40% identity between SEQ ID NOs:80 and 201.

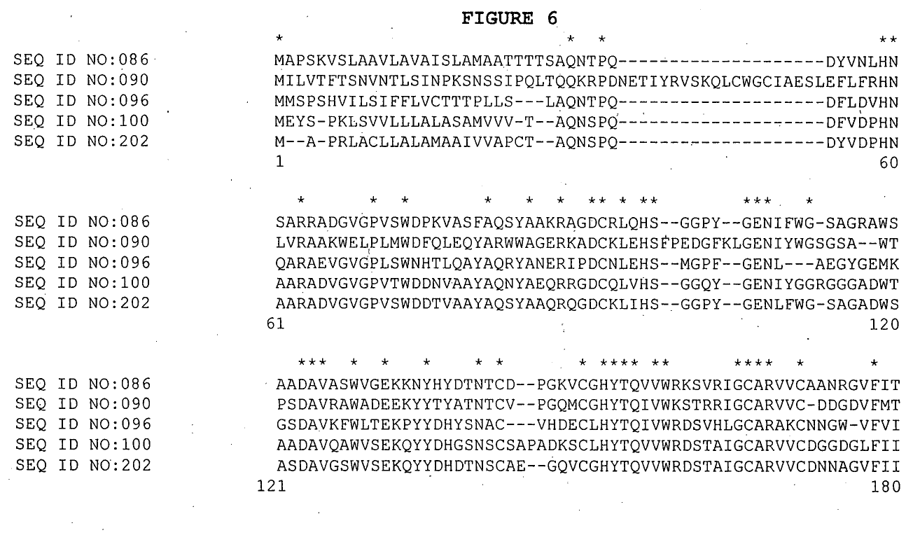

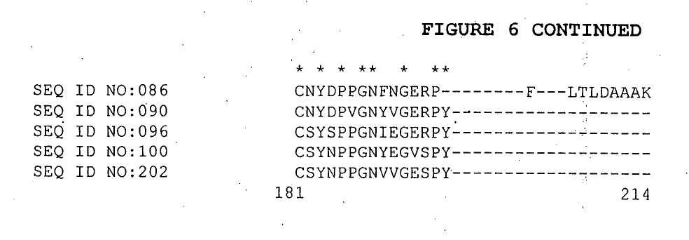

FIG. 6 depicts the amino acid sequence alignment between the pathogenesis-related (PR) protein encoded by the nucleotide sequences derived from rice clone rrl.pk077.e22 (SEQ ID NO:86), soybean clone sdp4c.pk009.g7 (SEQ ID NO:90), soybean clone src2c.pk023.b14 (SEQ ID NO:96), and wheat clone wlm96.pk025.j5 (SEQ ID NO:100), and the pathogenesis-related (PR) protein from Zea mays (NCBI GenBank Identifier (GI) No. 3290004; SEQ ID NO:202). Amino acids which are conserved among all and at least two sequences with an amino acid at that position are indicated with an asterisk (*). Dashes are used by the program to maximize alignment of the sequences. There is 63% identity between SEQ ID NOs:86 and 202, 71% identity between SEQ ID NO:86 and NCBI GI No. 1076732, 36% identity between SEQ ID NOs:90 and 202, 45% identity between SEQ ID NO:90 and NCBI GI No.2500715, 46% identity between SEQ ID NOs:96 and 202, 50% identity between SEQ ID NO:96 and NCBI GI No.130846, and 66% identity between SEQ ID NOs:100 and 202.

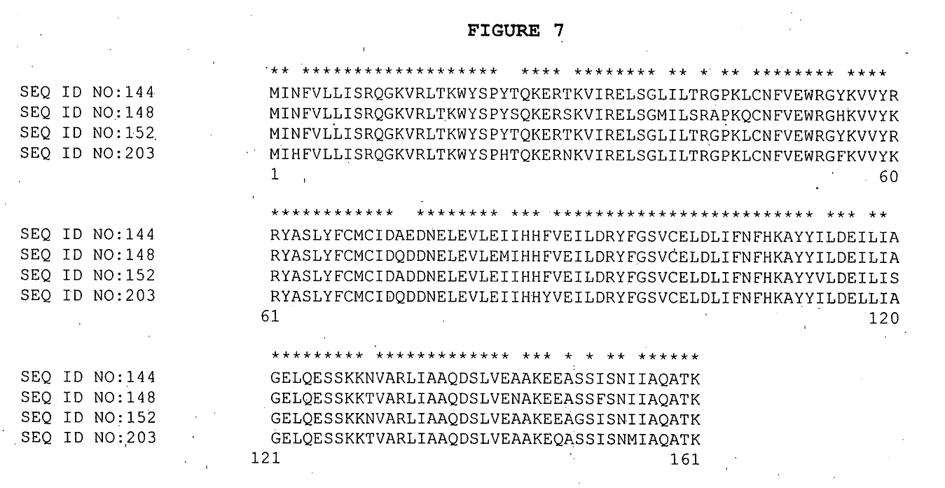

FIG. 7 depicts the amino acid sequence alignment between the AP19 protein encoded by the nucleotide sequences derived from corn clone p0038.crvak82r (SEQ ID NO:144), soybean clone srrlc.pk002.p3 (SEQ ID NO:148), and wheat clone wr1.pk148.a5 (SEQ ID NO:152), and the AP19 protein from Camptotheca acuminata (NCBI GenBank Identifier (GI) No. 1762309; SEQ ID NO:203). Amino acids which are conserved among all and at least two sequences with an amino acid at that position are indicated with an asterisk (*). Dashes are used by the program to maximize alignment of the sequences. There is 92% identity between SEQ ID NOs:144 and 203, 90% identity between SEQ ID NOs:148 and 203, and 91% identity between SEQ ID NOs:152 and 203.

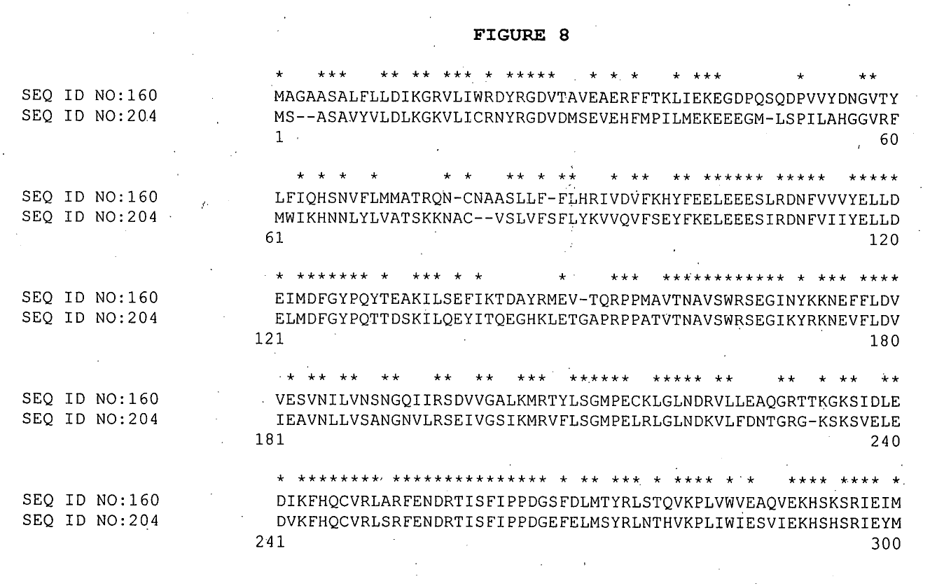

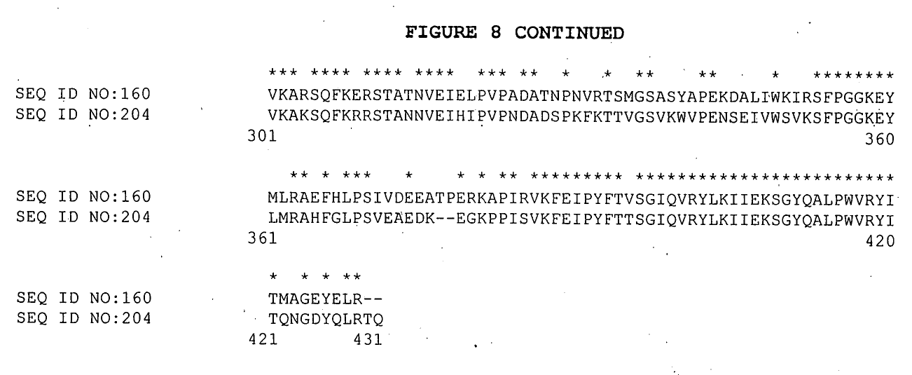

FIG. 8 depicts the amino acid sequence alignment between the AP47 protein encoded by the nucleotide sequence derived from soybean clone srrlc.pk003.g1 (SEQ ID NO:160) and the AP47 protein from Mus musculus (NCBI GenBank Identifier (GI) No. 6671557; SEQ ID NO:204). Amino acids which are conserved between the two sequences are indicated with an asterisk (*). Dashes are used by the program to maximize alignment of the sequences. There is 57% identity between SEQ ID NOs:160 and 204.

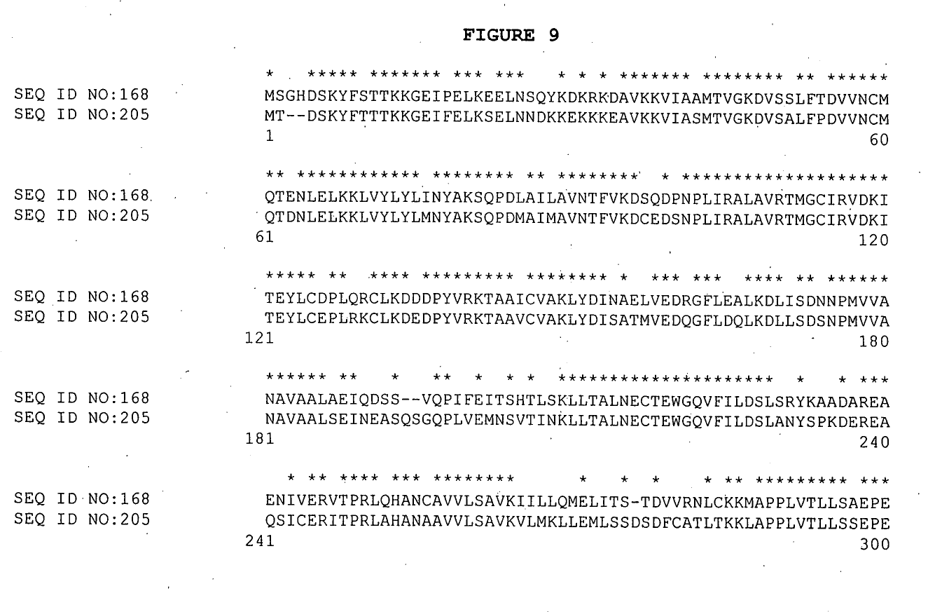

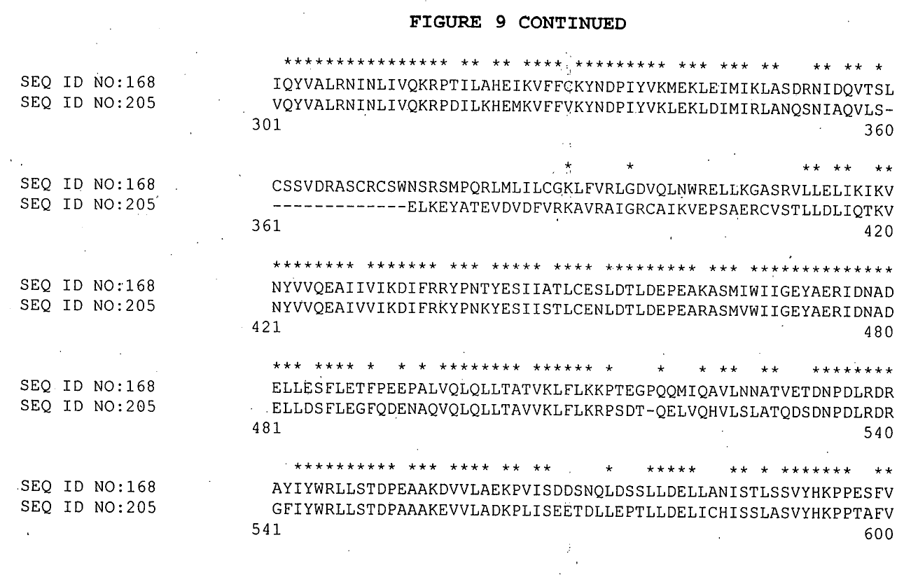

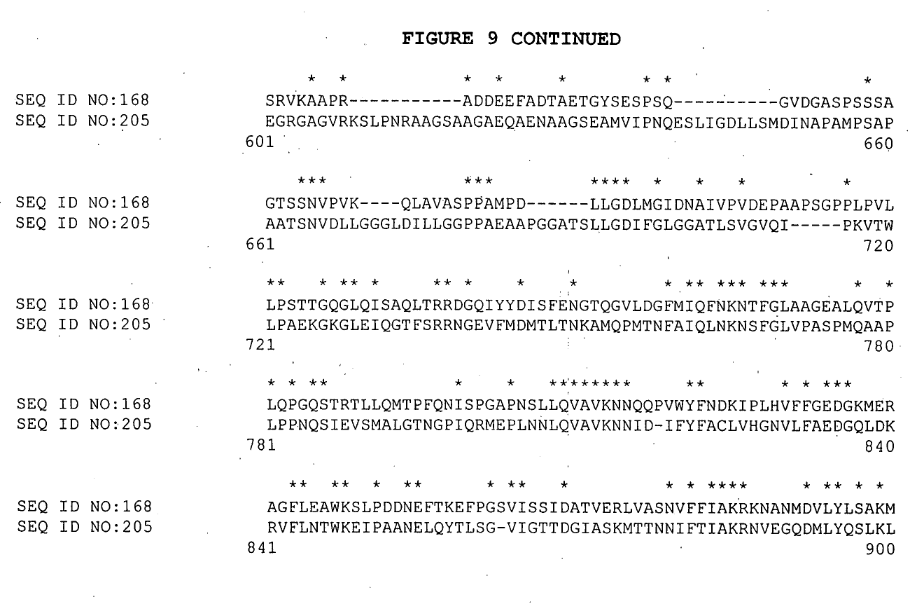

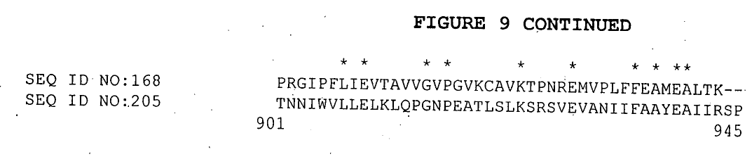

FIG. 9 depicts the amino acid sequence alignment between the beta-adaptin protein encoded by the nucleotide sequences derived from corn clone p019.cmtnr87r (SEQ ID NO:168) and the beta-adaptin protein from Drosophila melanogaster (NCBI GenBank Identifier (GI) No. 481762; SEQ ID NO:205). Amino acids which are conserved between the two sequences are indicated with an asterisk (*). Dashes are used by the program to maximize alignment of the sequences. There is 47% identity between SEQ ID NOs:168 and 205.

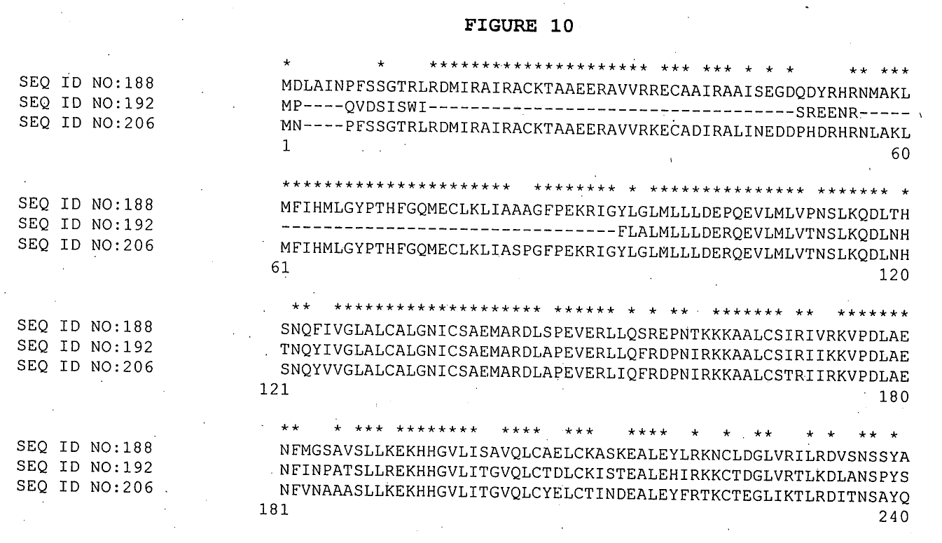

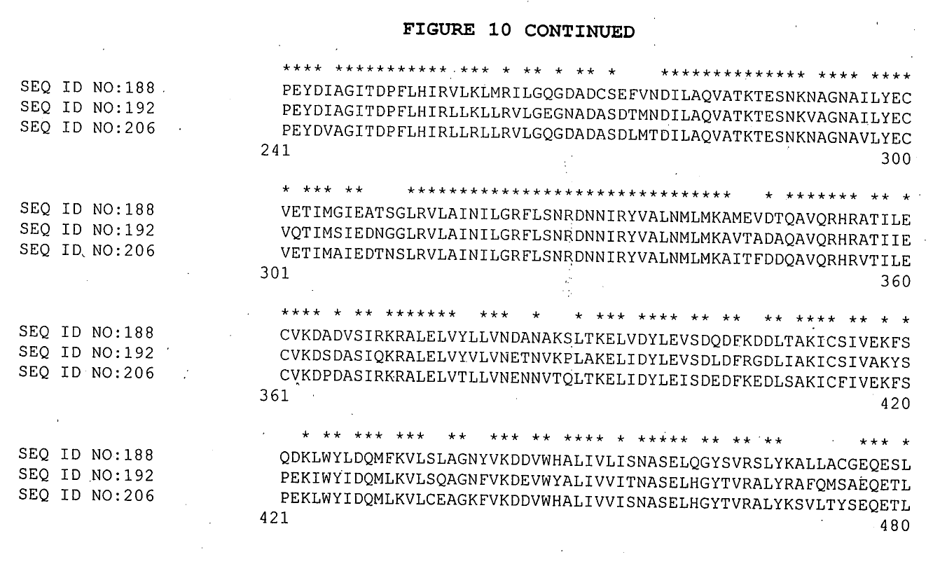

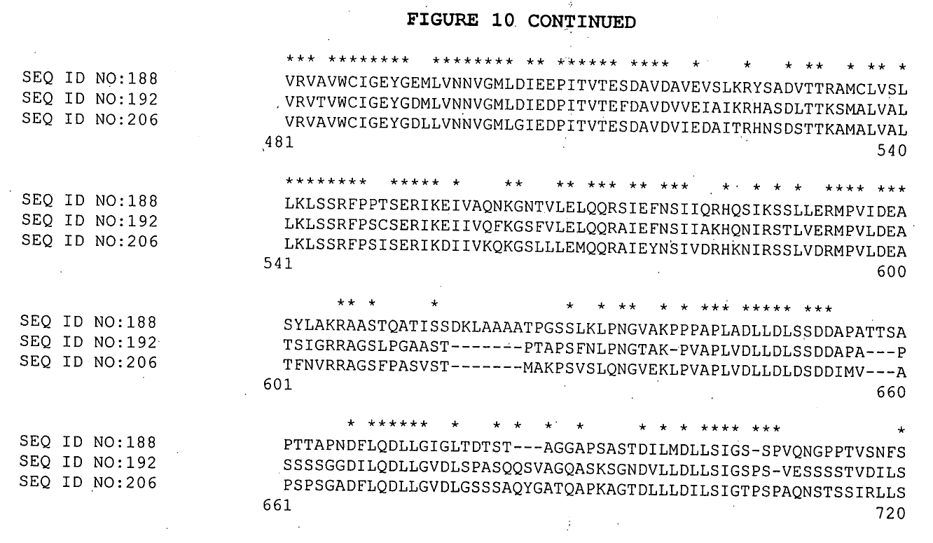

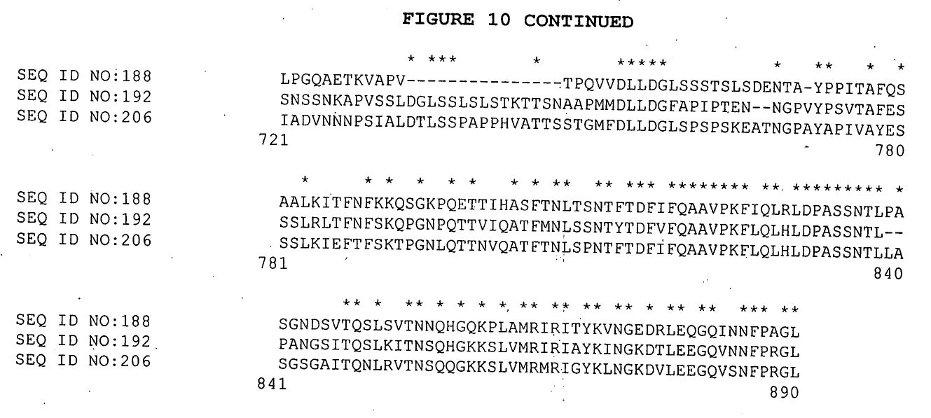

FIG. 10 depicts the amino acid sequence alignment between the gamma-adaptin protein encoded by the nucleotide sequences derived from rice clone rlr24.pk0087.a2 (SEQ ID NO:188) and soybean clone sgs4c.pk001j2 (SEQ ID NO:192), and the gamma-adaptin protein from Arabidopsis thaliana (NCBI GenBank Identifier (GI) No. 4538987; SEQ ID NO:206). Amino acids which are conserved among all and at least two sequences with an amino acid at that position are indicated with an asterisk (*). Dashes are used by the program to maximize alignment of the sequences. There is 66% identity between SEQ ID NOs:188 and 206, and 71% identity between SEQ ID NOs:192 and 206.

Sequence alignments and percent identity calculations were performed using the Megalign program of the LASERGENE bioinformatics computing suite (DNASTAR Inc., Madison, Wis.). Multiple alignment of the sequences was performed using the CLUSTAL method of alignment (Higgins and Sharp (1989) CABIOS. 5:151-153) with the default parameters (GAP PENALTY=10, GAP LENGTH PENALTY=10). Default parameters for pairwise alignments using the CLUSTAL method are KTUPLE 1, GAP PENALTY=3, WINDOW=5 and DIAGONALS SAVED=5.

B. Exemplary Utility of the Present Invention

The present invention provides utility in such exemplary applications as: developing strategies to improve plant response to stress, engineering plants with increased disease and stress resistance, manipulating DNA repair and recombination efficiency, manipulating intracellular protein transport, and improving/protecting grain flavor.

C. Exemplary Preferable Embodiments

While the various preferred embodiments are disclosed throughout the specification, exemplary preferable embodiments include the following:

(i) cDNA libraries representing mRNAs from various corn (Zea mays), rice (Oryza sativa), soybean (Glycine max), and wheat (Triticum aestivum) tissues were prepared. The characteristics of the libraries are described below.

TABLE 3
cDNA Libraries from Corn¹, Rice, Soybean, and Wheat

| Library | Tissue | Clone |
|---|---|---|
| chp2 | Corn (B73 and MK593) 11 Day Old Leaf Treated 24 Hours | chp2.pk0001.b11 |
| | With Herbicides² | |
| cpc1c | Corn pooled BMS treated with chemicals related to cGMP³ | cpc1c.pk004.o20 |
| cr1 | Corn Root From 7 Day Old Seedlings | cr1.pk0032.a11 |
| cr1n | Corn Root From 7 Day Old Seedlings⁴ | cr1n.pk0177.d10 |
| ctn1c | Corn Tassel, Night Harvested | ctn1c.pk001.i10 |
| p0010 | Corn Log Phase Suspension Cells Treated With A23187 ®⁵ | p0010.cbpbx77r |
| | to Induce Mass Apoptosis | p0010.cbpbx77rx |
| | | p0010.cbpcq26r |
| p0034 | Corn Endosperm 35 Days After Pollination | p0034.cdnad43r |
| p0037 | Corn V5 Stage Roots Infested With Corn Root Worm | p0037.crwaw93rb |
| p0038 | Corn V5-Stage Roots | p0038.crvak82r |
| p0050 | Corn Mid Rib from the Middle 3/4 of the 3rd Leaf Blade | p0050.cjlaa26r |
| | from Green Leaves Treated with Jasmonic Acid (1 mg/ml in | |
| | 0.02% Tween 20) 24 Hours Before Collection | |
| p0062 | Corn Coenocytic (4 Days After Pollination) Embryo Sacs | p0062.cymaj36r |
| p0084 | Corn Log Phase Suspension Cells Treated With A23187 ®⁵ | p0084.clopa50r |
| | to Induce Mass Apoptosis⁴ | |
| p0113 | Inner Layer of Endosperm (Starchy Endosperm)⁴ | p0113.cieab53r |
| p0119 | Corn V12-Stage Ear Shoot With Husk, Night Harvested⁴ | p0119.cmtnr87r |
| | | p0119.cmtoc10r |
| p0130 | Corn Wild-type Internode Tissue | p0130.cwtab66r |
| rca1c | Rice Nipponbare Callus | rca1c.pk005.116 |
| res1c | Rice Etiolated Seedling | res1c.pk007.h24 |
| rlr2 | Resistant Rice Leaf 15 Days After Germination, 2 Hours | rlr2.pk0020.g3 |
| | After Infection of Strain Magnaporthe grisea 4360-R-62 | |
| | (AVR2-YAMO) | |
| rlr24 | Resistant Rice Leaf 15 Days After Germination, 24 Hours | rlr24.pk0087.a2 |
| | After Infection of Strain Magnaporthe grisea 4360-R-62 | |
| | (AVR2-YAMO) | |
| rlr48 | Resistant Rice Leaf 15 Days After Germination, 48 Hours | rlr48.pk0007.c9 |
| | After Infection of Strain Magnaporthe grisea 4360-R-62 | |
| | (AVR2-YAMO) | |
| rlr6 | Resistant Rice Leaf 15 Days After Germination, 6 Hours | rlr6.pk0059.e10 |
| | After Infection of Strain Magnaporthe grisea 4360-R-62 | |
| | (AVR2-YAMO) | |
| rls6 | Susceptible Rice Leaf 15 Days After Germination, 6 Hours | rls6.pk0002.d12 |
| | After Infection of Strain Magnaporthe grisea 4360-R-67 | rls6.pk0084.f4 |
| | (AVR2-YAMO) | |
| rls72 | Susceptible Rice Leaf 15 Days After Germination, 72 Hours | rls72.pk0017.g8 |
| | After Infection of Strain Magnaporthe grisea 4360-R-67 | |
| | (AVR2-YAMO) | |
| rr1 | Rice Root of Two Week Old Developing Seedling | rr1.pk0024.g1 |
| | | rr1.pk077.e22 |
| | | rr1.pk079.o8 |
| sdp2c | Soybean Developing Pods (6-7 mm) | sdp2c.pk009.k16 |
| sdp3c | Soybean Developing Pods (8-9 mm) | sdp3c.pk016.115 |
| sdp4c | Soybean Developing Pods (10-12 mm) | sdp4c.pk001.o13 |
| | | sdp4c.pk009.g7 |
| se4 | Soybean Embryo, 19 Days After Flowering | se4.pk0018.e4 |
| sfl1 | Soybean Immature Flower | sfl1.pk126.j22 |
| sgs2c | Soybean Seeds 14 Hours After Germination | sgs2c.pk003.h9 |
| sgs4c | Soybean Seeds 2 Days After Germination | sgs4c.pk001.j2 |
| sgs5c | Soybean Seeds 4 Days After Germination | sgs5c.pk0003.e10 |
| sl2 | Soybean Two-Week-Old Developing Seedlings Treated With | sl2.pk0004.h5 |
| | 2.5 ppm chlorimuron | |
| sls1c | Soybean (S1990) Infected With Sclerotinia sclerotiorum | sls1c.pk010.p21 |
| | Mycelium | sls1c.pk024.m18 |
| sml1c | Soybean Mature Leaf | sml1c.pk004.n9 |
| sr1 | Soybean Root | sr1.pk0076.b9 |
| src2c | Soybean 8 Day Old Root Infected With Eggs of Cyst | src2c.pk004.f18 |
| | Nematode (Heteroderea glycensis) (Race 1) for 4 Days | src2c.pk023.b14 |
| | | src2c.pk028.d15 |
| src3c | Soybean 8 Day Old Root Infected With Cyst Nematode | src3c.pk020.h17 |
| | | src3c.pk022.p10 |
| srn1c | Soybean Developing Root Nodules | srn1c.pk002.c19 |
| srr1c | Soybean 8-Day-Old Root | srr1c.pk002.p3 |
| | | srr1c.pk003.g1 |
| ssm | Soybean Shoot Meristem | ssm.pk0011.h11 |
| wl1n | Wheat Leaf From 7 Day Old Seedling Light Grown⁴ | wl1n.pk0038.d10 |
| wlm0 | Wheat Seedlings 0 Hour After Inoculation With Erysiphe | wlm0.pk0014.a2 |
| | graminis f. sp tritici | |
| wlm96 | Wheat Seedlings 96 Hours After Inoculation With Erysiphe | wlm96.pk0001.b7 |
| | graminis f. sp tritici | wlm96.pk025.j5 |
| | | wlm96.pk028.k4 |
| | | wlm96.pk046.n12 |
| wlmk1 | Wheat Seedlings 1 Hour After Inoculation With Erysiphe | wlmk1.pk020.h8 |
| | graminis f. sp tritici and Treatment With Herbicide⁶ | |
| wlmk8 | Wheat Seedlings 8 Hours After Inoculation With Erysiphe | wlmk8.pk0021.e3 |
| | graminis f. sp tritici and Treatment With Herbicide⁶ | wlmk8.pk0022.c11 |
| wr1 | Wheat Root From 7 Day Old Seedling Light Grown | wr1.pk0018.g3 |
| | | wr1.pk148.a5 |
| wre1n | Wheat Root From 7 Day Old Etiolated Seedling⁴ | wre1n.pk0042.e2 |
| | | wre1n.pk0123.c3 |
| ¹Corn developmental stages are explained in the publication “How a corn plant develops” from the Iowa State University Coop. Ext. Service Special Report No. 48 reprinted June 1993. | | |
| ²Application of 2-[(2,4-dihydro-2,6,9-trimethyl[1]benzothiopyrano[4,3-c]pyrazol-8-yl)carbonyl]-1,3-cyclohexanedione S,S-dioxide (synthesis and methods of using this compound are described in WO 97/19087, incorporated herein by reference) and 2-[(2,3-dihydro-5,8-dimethylspiro[4H-1-benzothiopyran-4,2′-[1,3]dioxolan]-6-yl)carbonyl]-1,3-cyclohexanedione S,S-dioxide; also named 2-[(2,3-dihydro-5,8-dimethylspiro[4H-1- | | |
| # benzothiopyran-4,2′-[1,3]dioxolan]-6-yl)carbonyl]-3-hydroxy-2-cyclohexen-1-one S,S-dioxide (synthesis and methods of using this compound are described in WO 97/01550, incorporated herein by reference) | | |

³Chemicals used included suramin, MAS7, dipyryridamole, zaprinast, 8-bromo cGMP, trequinsin HCl, compound 48/80, all of which are commercially available from Calbiochem-Novabiochem Corp. (1-800-628-8470)
⁴These libraries were normalized essentially as described in U.S. Pat. No. 5,482,845, incorporated herein by reference.
⁵A23187 ® is commercially available from several vendors including Calbiochem (1-800-628-8470).
⁶Application of 6-iodo-2-propoxy-3-propyl-4(3H)-quinazolinone (synthesis and methods of using this compound are described in USSN 08/545,827, incorporated herein by reference)

cDNA libraries may be prepared by any one of many methods available. For example, the cDNAs may be introduced into plasmid vectors by first preparing the cDNA libraries in Uni-ZAP™ XR vectors according to the manufacturer's protocol (Stratagene Cloning Systems, La Jolla, Calif.). The Uni-ZAP™ XR libraries are converted into plasmid libraries according to the protocol provided by Stratagene. Upon conversion, cDNA inserts will be contained in the plasmid vector pBluescript. In addition, the cDNAs may be introduced directly into precut Bluescript II SK(+) vectors (Stratagene) using T4 DNA ligase (New England Biolabs), followed by transfection into DH10B cells according to the manufacturer's protocol (GIBCO BRL Products). Once the cDNA inserts are in plasmid vectors, plasmid DNAs are prepared from randomly picked bacterial colonies containing recombinant pBluescript plasmids, or the insert cDNA sequences are amplified via polymerase chain reaction using primers specific for vector sequences flanking the inserted cDNA sequences. Amplified insert DNAs or plasmid DNAs are sequenced in dye-primer sequencing reactions to generate partial cDNA sequences (expressed sequence tags or “ESTs”; see Adams et al., (1991) Science 252:1651-1656). The resulting ESTs are analyzed using a Perkin Elmer Model 377 fluorescent sequencer.

Definitions

Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges recited within the specification are inclusive of the numbers defining the range and include each integer within the defined range. Amino acids may be referred to herein by either their commonly known three letter symbols or by the one-letter symbols recommended by the IUPAC-IUBMB Nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5^{th} edition, 1993). The terms defined below are more fully defined by reference to the specification as a whole. Section headings provided throughout the specification are not limitations to the various objects and embodiments of the present invention.

“Stress response protein” refers to a protein that is involved in enabling the plant to respond to biotic and abiotic stresses. A stress response protein may not be directly involved in the stress response but is needed to effect a particular stress response.

“Amplified” refers to the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

As used herein, “antisense orientation” includes reference to a duplex polynucleotide sequence that is operably linked to a promoter in an orientation where the antisense strand is transcribed. The antisense strand is sufficiently complementary to an endogenous transcription product such that translation of the endogenous transcription product is often inhibited.

“Encoding” or “encoded”, with respect to a specified nucleic acid, refers to comprising the information for translation into the specified protein. A nucleic acid encoding a protein may comprise intervening sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fungal mitochondria, the bacterium Mycoplasma capricolum, or the ciliate Macronucleus, may be used when the nucleic acid is expressed therein.

When the nucleic acid is prepared or altered synthetically, advantage can be taken of known codon preferences of the intended host in which the nucleic acid is to be expressed. For example, although nucleic acid sequences of the present invention may be expressed in both monocotyledonous and dicotyledonous plant species, sequences can be modified to account for the specific codon preferences and GC content preferences of monocotyledons or dicotyledons as these preferences have been shown to differ (Murray et al., Nuc. Acids Res. 17:477-498 (1989)). Thus, the maize preferred codon for a particular amino acid may be derived from known gene sequences from maize. Maize codon usage for 28 genes from maize plants is listed in Table 4 of Murray et al., supra.

As used herein “full-length sequence” in reference to a specified polynucleotide or its encoded protein means having the entire amino acid sequence of, a native (non-synthetic), endogenous, biologically (e.g., structurally or catalytically) active form of the specified protein. Methods to determine whether a sequence is full-length are well known in the art including such exemplary techniques as Northern or Western blots, primer extension, SI protection, and ribonuclease protection. See, e.g., Plant Molecular Biology: A Laboratory Manual, Clark, Ed., Springer-Verlag, Berlin (1997). Comparison to known full-length homologous (orthologous and/or paralogous) sequences can also be used to identify full-length sequences of the present invention. Additionally, consensus sequences typically present at the 5′ and 3′ untranslated regions of mRNA aid in the identification of a polynucleotide as full-length. For example, the consensus sequence ANNNNAUGG, where the underlined codon represents the N-terminal methionine, aids in determining whether the polynucleotide has a complete 5′ end. Consensus sequences at the 3′ end, such as polyadenylation sequences, aid in determining whether the polynucleotide has a complete 3′ end.

As used herein, “heterologous”, in reference to a nucleic acid is a nucleic acid that originates from a foreign species, or, if from the same species, is substantially modified from its native form in composition and/or genomic locus by human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by human intervention.

“Host cell” refers to a cell which contains a vector and supports the replication and/or expression of the vector. Host cells may be prokaryotic cells such as E. coli, or eukaryotic cells such as yeast, insect, amphibian, or mammalian cells. Preferably, host cells are monocotyledonous or dicotyledonous plant cells. A particularly preferred monocotyledonous host cell is a maize host cell.

The term “introduced” includes reference to the incorporation of a nucleic acid into a eukaryotic or prokaryotic cell wherein the nucleic acid may be incorporated into the genome of the cell (e.g., chromosome, plasmid, plastid or mitochondrial DNA), converted into an autonomous replicon, or transiently expressed (e.g., transfected mRNA). The term includes such nucleic acid introduction means as “transfection”, “transformation” and “transduction”.

The term “isolated” refers to material, such as a nucleic acid or a protein, which is substantially free from components that normally accompany or interact with it as found in its naturally occurring environment. The isolated material optionally comprises material not found with the material in its natural environment, or if the material is in its natural environment, the material has been synthetically (non-naturally) altered by human intervention to a composition and/or placed at a location in the cell (e.g., genome or subcellular organelle) not native to a material found in that environment. The alteration to yield the synthetic material can be performed on the material within or removed from its natural state. For example, a naturally occurring nucleic acid becomes an isolated nucleic acid if it is altered, or if it is transcribed from DNA which has been altered, by means of human intervention performed within the cell from which it originates. See, e.g., Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868. Likewise, a naturally occurring nucleic acid (e.g., a promoter) becomes isolated if it is introduced by non-naturally occurring means to a locus of the genome not native to that nucleic acid. Nucleic acids which are “isolated” as defined herein, are also referred to as “heterologous” nucleic acids.

As used herein, “nucleic acid” includes reference to a deoxyribonucleotide or ribonucleotide polymer, or chimeras thereof, in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids). “Nucleic acid library” refers to a collection of isolated DNA or RNA molecules which comprise and substantially represent the entire transcribed fraction of a genome of a specified organism, tissue, or of a cell type from that organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, Calif. (Berger); Sambrook et al., Molecular Cloning—A Laboratory Manual, 2nd ed., Vol. 1-3 (1989); and Current Protocols in Molecular Biology, F. M. Ausubel et al., Eds., Current Protocols, a joint venture between Greene Publishing Associates, Inc. and John Wiley & Sons, Inc. (1994).

As used herein “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen, and microspores. The class of plants which can be used in the methods of the invention include both monocotyledonous and dicotyledonous plants. A particularly preferred plant is Zea mays.

As used herein, “polynucleotide” includes reference to a deoxyribopolynucleotide, ribopolynucleotide, or chimeras or analogs thereof that have the essential nature of a natural deoxy- or ribo-nucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are “polynucleotides” as the term is used herein. It will be appreciated that a great variety of modifications have been made to DNA and RNA that serve many useful purposes known to those of skill in the art. The term “polynucleotide” as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. Further, this invention contemplates the use of both the methionine-containing and the methionine-less amino terminal variants of the protein of the invention.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells whether or not its origin is a plant cell. Exemplary plant promoters include, but are not limited to, those that are obtained from plants, plant viruses, and bacteria which comprise genes expressed in plant cells such Agrobacterium or Rhizobium. Examples of promoters under developmental control include promoters that preferentially initiate transcription in certain tissues, such as leaves, roots, or seeds. Such promoters are referred to as “tissue preferred”. Promoters which initiate transcription only in certain tissue are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may effect transcription by inducible promoters include anaerobic conditions or the presence of light. Tissue specific, tissue preferred, cell type specific, and inducible promoters constitute the class of “non-constitutive” promoters. A “constitutive” promoter is a promoter which is active under most environmental conditions.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of human intervention. The term “recombinant” as used herein does not encompass the alteration of the cell or vector by naturally occurring events (e.g., spontaneous mutation, natural transformation/transduction/transposition) such as those occurring without human intervention.

As used herein, a “recombinant expression cassette” is a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The term “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, preferably 90% sequence identity, and most preferably 100% sequence identity (i.e., complementary) with each other.

The term “stringent conditions” or “stringent hybridization conditions” includes reference to conditions under which a probe will selectively hybridize to its target sequence, to a detectably greater degree than to other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 110C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. Hybridization and/or wash conditions can be applied for at least 10, 30, 60, 90, 120, or 240 minutes. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel, et al., Eds., Greene Publishing and Wiley-Interscience, New York (1995).

As used herein, “transgenic plant” includes reference to a plant which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, “vector” includes reference to a nucleic acid used in the introduction of a polynucleotide of the present invention into a host cell. Vectors are often replicons. Expression vectors permit transcription of a nucleic acid inserted therein.

The following terms are used to describe the sequence relationships between a polynucleotide/polypeptide of the present invention with a reference polynucleotide/polypeptide: (a) “reference sequence”, (b) “comparison window”, (c) “sequence identity”, and (d) “percentage of sequence identity”.

(a) As used herein, “reference sequence” is a defined sequence used as a basis for sequence comparison with a polynucleotide/polypeptide of the present invention. A reference sequence may be a subset or the entirety of a specified sequence; for example, a segment of a full-length cDNA or gene sequence, or the complete cDNA or gene sequence.

(b) As used herein, “comparison window” includes reference to a contiguous and specified segment of a polynucleotide/polypeptide sequence, wherein the polynucleotide/polypeptide sequence may be compared to a reference sequence and wherein the portion of the polynucleotide/polypeptide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides/amino acids residues in length, and optionally can be 30, 40, 50, 100, or longer. Those of skill in the art understand that to avoid a high similarity to a reference sequence due to inclusion of gaps in the polynucleotide/polypeptide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16: 10881-90 (1988); Huang, et al., Computer Applications in the Biosciences 8:155-65 (1992), and Pearson, et al., Methods in Molecular Biology 24:307-331 (1994).

The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995); Altschul et al., J. Mol. Biol., 215:403-410 (1990); and, Altschul et al., Nucleic Acids Res. 25:3389-3402 (1997).

Software for performing BLAST analyses is publicly available, e.g., through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff& Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5877 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Clayerie and States, Comput. Chem., 17:191-201 (1993)) low-complexity filters can be employed alone or in combination.

Unless otherwise stated, nucleotide and protein identity/similarity values provided herein are calculated using GAP (GCG Version 10) under default values.

GAP (Global Alignment Program) can also be used to compare a polynucleotide or polypeptide of the present invention with a reference sequence. GAP uses the algorithm of Needleman and Wunsch (J. Mol. Biol. 48:443-453, 1970) to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 100. Thus, for example, the gap creation and gap extension penalties can each independently be: 0, 1, 2, 3, 4, 5, 6, 7, 8, 9, 10, 15, 20, 30, 40, 50, 60 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff & Henikoff(1989) Proc. Natl. Acad. Sci. USA 89:10915).

Multiple alignment of the sequences can be performed using the CLUSTAL method of alignment (Higgins and Sharp (1989) CABIOS. 5:151-153) with the default parameters (GAP PENALTY=10, GAP LENGTH PENALTY=10). Default parameters for pairwise alignments using the CLUSTAL method are KTUPLE 1, GAP PENALTY=3, WINDOW=5 and DIAGONALS SAVED=5.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4: 11-17 (1988) e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

Utilities

The present invention provides, among other things, compositions and methods for modulating (i.e., increasing or decreasing) the level of polynucleotides and polypeptides of the present invention in plants. In particular, the polynucleotides and polypeptides of the present invention can be expressed temporally or spatially, e.g., at developmental stages, in tissues, and/or in quantities, which are uncharacteristic of non-recombinantly engineered plants.

The present invention also provides isolated nucleic acids comprising polynucleotides of sufficient length and complementarity to a polynucleotide of the present invention to use as probes or amplification primers in the detection, quantitation, or isolation of gene transcripts. For example, isolated nucleic acids of the present invention can be used as probes in detecting deficiencies in the level of mRNA in screenings for desired transgenic plants, for detecting mutations in the gene (e.g., substitutions, deletions, or additions), for monitoring upregulation of expression or changes in enzyme activity in screening assays of compounds, for detection of any number of allelic variants (polymorphisms), orthologs, or paralogs of the gene, or for site directed mutagenesis in eukaryotic cells (see, e.g., U.S. Pat. No. 5,565,350). The isolated nucleic acids of the present invention can also be used for recombinant expression of their encoded polypeptides, or for use as immunogens in the preparation and/or screening of antibodies. The isolated nucleic acids of the present invention can also be employed for use in sense or antisense suppression of one or more genes of the present invention in a host cell, tissue, or plant. Attachment of chemical agents which bind, intercalate, cleave and/or crosslink to the isolated nucleic acids of the present invention can also be used to modulate transcription or translation.

The present invention also provides isolated proteins comprising a polypeptide of the present invention (e.g., preproenzyme, proenzyme, or enzymes). The present invention also provides proteins comprising at least one epitope from a polypeptide of the present invention. The proteins of the present invention can be employed in assays for enzyme agonists or antagonists of enzyme function, or for use as immunogens or antigens to obtain antibodies specifically immunoreactive with a protein of the present invention. Such antibodies can be used in assays for expression levels, for identifying and/or isolating nucleic acids of the present invention from expression libraries, for identification of homologous polypeptides from other species, or for purification of polypeptides of the present invention.

The isolated nucleic acids and polypeptides of the present invention can be used over a broad range of plant types, particularly monocots such as the species of the family Gramineae including Hordeum, Secale, Oryza, Triticum, Sorghum (e.g., S. bicolor) and Zea (e.g., Z. mays), and dicots such as Glycine.

The isolated nucleic acid and proteins of the present invention can also be used in species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browallia, Pisum, Phaseolus, Lolium, and Avena.

Nucleic Acids

The present invention provides, among other things, isolated nucleic acids of RNA, DNA, and analogs and/or chimeras thereof, comprising a polynucleotide of the present invention. A polynucleotide of the present invention is inclusive of those in Table 1 and:
(a) an isolated polynucleotide encoding a polypeptide of the present invention such as those referenced in Table 1, including exemplary polynucleotides of the present invention;
(b) an isolated polynucleotide which is the product of amplification from a plant nucleic acid library using primer pairs which selectively hybridize under stringent conditions to loci within a polynucleotide of the present invention;
(c) an isolated polynucleotide which selectively hybridizes to a polynucleotide of (a) or (b);
(d) an isolated polynucleotide having a specified sequence identity with polynucleotides of (a), (b), or (c);
(e) an isolated polynucleotide encoding a protein having a specified number of contiguous amino acids from a prototype polypeptide, wherein the protein is specifically recognized by antisera elicited by presentation of the protein and wherein the protein does not detectably immunoreact to antisera which has been fully immunosorbed with the protein;
(f) complementary polynucleotide sequences of (a), (b), (c), (d), or (e); and
(g) an isolated polynucleotide comprising at least a specific number of contiguous nucleotides from a polynucleotide of (a), (b), (c), (d), (e), or (f);
(h) an isolated polynucleotide from a full-length enriched cDNA library having the physico-chemical property of selectively hybridizing to a polynucleotide of (a), (b), (c), (d), (e), (f), or (g);
(i) an isolated polynucleotide made by the process of: 1) providing a full-length enriched nucleic acid library, 2) selectively hybridizing the polynucleotide to a polynucleotide of (a), (b), (c), (d), (e), (f), (g), or (h), thereby isolating the polynucleotide from the nucleic acid library.
   A. Polynucleotides Encoding A Polypeptide of the Present Invention

As indicated in (a), above, the present invention provides for isolated nucleic acids comprising a polynucleotide of the present invention, wherein the polynucleotide encodes a polypeptide of the present invention. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Thus, each silent variation of a nucleic acid which encodes a polypeptide of the present invention is implicit in each described polypeptide sequence and is within the scope of the present invention. Accordingly, the present invention includes polynucleotides of the present invention and polynucleotides encoding a polypeptide of the present invention.

B. Polynucleotides Amplified from a Plant Nucleic Acid Library

As indicated in (b), above, the present invention provides an isolated nucleic acid comprising a polynucleotide of the present invention, wherein the polynucleotides are amplified, under nucleic acid amplification conditions, from a plant nucleic acid library. Nucleic acid amplification conditions for each of the variety of amplification methods are well known to those of ordinary skill in the art. The plant nucleic acid library can be constructed from a monocot such as a cereal crop. Exemplary cereals include corn, sorghum, alfalfa, canola, wheat, or rice. The plant nucleic acid library can also be constructed from a dicot such as soybean. Zea mays lines B73, PHRE1, A632, BMS-P2#10, W23, and Mol 7 are known and publicly available. Other publicly known and available maize lines can be obtained from the Maize Genetics Cooperation (Urbana, Ill.). Wheat lines are available from the Wheat Genetics Resource Center (Manhattan, Kans.).

The nucleic acid library may be a cDNA library, a genomic library, or a library generally constructed from nuclear transcripts at any stage of intron processing. cDNA libraries can be normalized to increase the representation of relatively rare cDNAs. In optional embodiments, the cDNA library is constructed using an enriched full-length cDNA synthesis method. Examples of such methods include Oligo-Capping (Maruyama, K. and Sugano, S. Gene 138:171-174, 1994), Biotinylated CAP Trapper (Carninci, et al. Genomics 37:327-336, 1996), and CAP Retention Procedure (Edery, E., Chu, L. L., et al. Molecular and Cellular Biology 15:3363-3371, 1995). Rapidly growing tissues or rapidly dividing cells are preferred for use as an mRNA source for construction of a cDNA library. Growth stages of corn is described in “How a Corn Plant Develops,” Special Report No. 48, Iowa State University of Science and Technology Cooperative Extension Service, Ames, Iowa, Reprinted February 1993.

A polynucleotide of this embodiment (or subsequences thereof) can be obtained, for example, by using amplification primers which are selectively hybridized and primer extended, under nucleic acid amplification conditions, to at least two sites within a polynucleotide of the present invention, or to two sites within the nucleic acid which flank and comprise a polynucleotide of the present invention, or to a site within a polynucleotide of the present invention and a site within the nucleic acid which comprises it. Methods for obtaining 5′ and/or 3′ ends of a vector insert are well known in the art. See, e.g., RACE (Rapid Amplification of Complementary Ends) as described in Frohman, M. A., in PCR Protocols: A Guide to Methods and Applications, M. A. Innis, D. H. Gelfand, J. J. Sninsky, T. J. White, Eds. (Academic Press, Inc., San Diego), pp. 28-38 (1990)); see also, U.S. Pat. No. 5,470,722, and Current Protocols in Molecular Biology, Unit 15.6, Ausubel, et al., Eds., Greene Publishing and Wiley-Interscience, New York (1995); Frohman and Martin, Techniques 1:165 (1989).

Optionally, the primers are complementary to a subsequence of the target nucleic acid which they amplify but may have a sequence identity ranging from about 85% to 99% relative to the polynucleotide sequence which they are designed to anneal to. As those skilled in the art will appreciate, the sites to which the primer pairs will selectively hybridize are chosen such that a single contiguous nucleic acid can be formed under the desired nucleic acid amplification conditions. The primer length in nucleotides is selected from the group of integers consisting of from at least 15 to 50. Thus, the primers can be at least 15, 18, 20, 25, 30, 40, or 50 nucleotides in length. Those of skill will recognize that a lengthened primer sequence can be employed to increase specificity of binding (i.e., annealing) to a target sequence. A non-annealing sequence at the 5′end of a primer (a “tail”) can be added, for example, to introduce a cloning site at the terminal ends of the amplicon.

The amplification products can be translated using expression systems well known to those of skill in the art. The resulting translation products can be confirmed as polypeptides of the present invention by, for example, assaying for the appropriate catalytic activity (e.g., specific activity and/or substrate specificity), or verifying the presence of one or more epitopes which are specific to a polypeptide of the present invention. Methods for protein synthesis from PCR derived templates are known in the art and available commercially. See, e.g., Amersham Life Sciences, Inc, Catalog '97, p. 354.

C. Polynucleotides Which Selectively Hybridize to a Polynucleotide of (A) or (B)

As indicated in (c), above, the present invention provides isolated nucleic acids comprising polynucleotides of the present invention, wherein the polynucleotides selectively hybridize, under selective hybridization conditions, to a polynucleotide of sections (A) or (B) as discussed above. Thus, the polynucleotides of this embodiment can be used for isolating, detecting, and/or quantifying nucleic acids comprising the polynucleotides of (A) or (B). For example, polynucleotides of the present invention can be used to identify, isolate, or amplify partial or full-length clones in a deposited library. In some embodiments, the polynucleotides are genomic or cDNA sequences isolated or otherwise complementary to a cDNA from a dicot or monocot nucleic acid library. Exemplary species of monocots and dicots include, but are not limited to: maize, canola, soybean, cotton, wheat, sorghum, sunflower, alfalfa, oats, sugar cane, millet, barley, and rice. The cDNA library comprises at least 50% to 95% full-length sequences (for example, at least 50%, 60%, 70%, 80%, 90%, or 95% full-length sequences). The cDNA libraries can be normalized to increase the representation of rare sequences. See, e.g., U.S. Pat. No. 5,482,845. Low stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% to 80% sequence identity and can be employed to identify orthologous or paralogous sequences.

D. Polynucleotides Having a Specific Sequence Identity with the Polynucleotides of (A), (B) or (C)

As indicated in (d), above, the present invention provides isolated nucleic acids comprising polynucleotides of the present invention, wherein the polynucleotides have a specified identity at the nucleotide level to a polynucleotide as disclosed above in sections (A), (B), or (C), above. Identity can be calculated using, for example, the BLAST, CLUSTALW, or GAP algorithms under default conditions. The percentage of identity to a reference sequence is at least 60% and, rounded upwards to the nearest integer, can be expressed as an integer selected from the group of integers consisting of from 60 to 99. Thus, for example, the percentage of identity to a reference sequence can be at least 70%, 75%, 80%, 85%, 90%, or 95%.

Optionally, the polynucleotides of this embodiment will encode a polypeptide that will share an epitope with a polypeptide encoded by the polynucleotides of sections (A), (B), or (C). Thus, these polynucleotides encode a first polypeptide which elicits production of antisera comprising antibodies which are specifically reactive to a second polypeptide encoded by a polynucleotide of (A), (B), or (C). However, the first polypeptide does not bind to antisera raised against itself when the antisera has been fully immunosorbed with the first polypeptide. Hence, the polynucleotides of this embodiment can be used to generate antibodies for use in, for example, the screening of expression libraries for nucleic acids comprising polynucleotides of (A), (B), or (C), or for purification of, or in immunoassays for, polypeptides encoded by the polynucleotides of (A), (B), or (C). The polynucleotides of this embodiment comprise nucleic acid sequences which can be employed for selective hybridization to a polynucleotide encoding a polypeptide of the present invention.

Screening polypeptides for specific binding to antisera can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids long, and often from about 8 to 15 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT patent publication Nos. 91/17271, 91/18980, 91/19818, and 93/08278. Other systems for generating libraries of peptides have aspects of both in vitro chemical synthesis and recombinant methods. See, PCT Patent publication Nos. 92/05258, 92/14843, and 97/20078. See also, U.S. Pat. Nos. 5,658,754; and 5,643,768. Peptide display libraries, vectors, and screening kits are commercially available from such suppliers as Invitrogen (Carlsbad, Calif.).E. Polynucleotides Encoding a Protein Having a Subsequence from a Prototype Polypeptide and Cross-Reactive to the Prototype Polypeptide

As indicated in (e), above, the present invention provides isolated nucleic acids comprising polynucleotides of the present invention, wherein the polynucleotides encode a protein having a subsequence of contiguous amino acids from a prototype polypeptide of the present invention such as are provided in (a), above. The length of contiguous amino acids from the prototype polypeptide is selected from the group of integers consisting of from at least 10 to the number of amino acids within the prototype sequence. Thus, for example, the polynucleotide can encode a polypeptide having a subsequence having at least 10, 15, 20, 25, 30, 35, 40, 45, or 50, contiguous amino acids from the prototype polypeptide. Further, the number of such subsequences encoded by a polynucleotide of the instant embodiment can be any integer selected from the group consisting of from 1 to 20, such as 2, 3, 4, or 5. The subsequences can be separated by any integer of nucleotides from 1 to the number of nucleotides in the sequence such as at least 5, 10, 15, 25, 50, 100, or 200 nucleotides.

The proteins encoded by polynucleotides of this embodiment, when presented as an immunogen, elicit the production of polyclonal antibodies which specifically bind to a prototype polypeptide such as but not limited to, a polypeptide encoded by the polynucleotide of (a) or (b), above. Generally, however, a protein encoded by a polynucleotide of this embodiment does not bind to antisera raised against the prototype polypeptide when the antisera has been fully immunosorbed with the prototype polypeptide. Methods of making and assaying for antibody binding specificity/affinity are well known in the art Exemplary immunoassay formats include ELISA, competitive immunoassays, radioimmunoassays, Western blots, indirect immunofluorescent assays and the like.

In a preferred assay method, fully immunosorbed and pooled antisera which is elicited to the prototype polypeptide can be used in a competitive binding assay to test the protein. The concentration of the prototype polypeptide required to inhibit 50% of the binding of the antisera to the prototype polypeptide is determined. If the amount of the protein required to inhibit binding is less than twice the amount of the prototype protein, then the protein is said to specifically bind to the antisera elicited to the immunogen. Accordingly, the proteins of the present invention embrace allelic variants, conservatively modified variants, and minor recombinant modifications to a prototype polypeptide.

A polynucleotide of the present invention optionally encodes a protein having a molecular weight as the non-glycosylated protein within 20% of the molecular weight of the full-length non-glycosylated polypeptides of the present invention. Molecular weight can be readily determined by SDS-PAGE under reducing conditions. Optionally, the molecular weight is within 15% of a full length polypeptide of the present invention, more preferably within 10% or 5%, and most preferably within 3%, 2%, or 1% of a full length polypeptide of the present invention.

Optionally, the polynucleotides of this embodiment will encode a protein having a specific enzymatic activity at least 50%, 60%, 80%, or 90% of a cellular extract comprising the native, endogenous full-length polypeptide of the present invention. Further, the proteins encoded by polynucleotides of this embodiment will optionally have a substantially similar affinity constant (Kₘ) and/or catalytic activity (i.e., the microscopic rate constant, k_{cat}) as the native endogenous, full-length protein. Those skilled in the art will recognize that the k_{cat}/Kₘ value determines the specificity for competing substrates and is often referred to as the specificity constant. Proteins of this embodiment can have a k_{cat}/Kₘ value at least 10% of a full-length polypeptide of the present invention as determined using the endogenous substrate of that polypeptide. Optionally, the k_{cat}/Kₘ value will be at least 20%, 30%, 40%, 50%, and most preferably at least 60%, 70%, 80%, 90%, or 95% of the k_{cat}/Kₘ value of the full-length polypeptide of the present invention. Determination of k_{cat}, Kₘ, and k_{cat}/Kₘ can be determined by any number of means well known to those of skill in the art. For example, the initial rates (i.e., the first 5% or less of the reaction) can be determined using rapid mixing and sampling techniques (e.g., continuous-flow, stopped-flow, or rapid quenching techniques), flash photolysis, or relaxation methods (e.g., temperature jumps) in conjunction with such exemplary methods of measuring as spectrophotometry, spectrofluorimetry, nuclear magnetic resonance, or radioactive procedures. Kinetic values are conveniently obtained using a Lineweaver-Burk or Eadie-Hofstee plot.

F. Polynucleotides Complementary to the Polynucleotides of (A)-(E)

As indicated in (f), above, the present invention provides isolated nucleic acids comprising polynucleotides complementary to the polynucleotides of paragraphs A-E, above. As those of skill in the art will recognize, complementary sequences base-pair throughout the entirety of their length with the polynucleotides of sections (A)-(E) (i.e., have 100% sequence identity over their entire length). Complementary bases associate through hydrogen bonding in double stranded nucleic acids. For example, the following base pairs are complementary: guanine and cytosine; adenine and thymine; and adenine and uracil.

G. Polynucleotides Which are Subsequences of the Polynucleotides of (A)-(F)

As indicated in (g), above, the present invention provides isolated nucleic acids comprising polynucleotides which comprise at least 15 contiguous bases from the polynucleotides of sections (A) through (F) as discussed above. The length of the polynucleotide is given as an integer selected from the group consisting of from at least 15 to the length of the nucleic acid sequence from which the polynucleotide is a subsequence of. Thus, for example, polynucleotides of the present invention are inclusive of polynucleotides comprising at least 15, 20, 25, 30, 40, 50, 60, 75, or 100 contiguous nucleotides in length from the polynucleotides of (A)-(F). Optionally, the number of such subsequences encoded by a polynucleotide of the instant embodiment can be any integer selected from the group consisting of from 1 to 20, such as 2, 3, 4, or 5. The subsequences can be separated by any integer of nucleotides from 1 to the number of nucleotides in the sequence such as at least 5, 10, 15, 25, 50, 100, or 200 nucleotides.

Subsequences can be made by in vitro synthetic, in vitro biosynthetic, or in vivo recombinant methods. In optional embodiments, subsequences can be made by nucleic acid amplification. For example, nucleic acid primers will be constructed to selectively hybridize to a sequence (or its complement) within, or co-extensive with, the coding region.

The subsequences of the present invention can comprise structural characteristics of the sequence from which it is derived. Alternatively, the subsequences can lack certain structural characteristics of the larger sequence from which it is derived such as a poly (An) tail. Optionally, a subsequence from a polynucleotide encoding a polypeptide having at least one epitope in common with a prototype polypeptide sequence as provided in (a), above, may encode an epitope in common with the prototype sequence. Alternatively, the subsequence may not encode an epitope in common with the prototype sequence but can be used to isolate the larger sequence by, for example, nucleic acid hybridization with the sequence from which it's derived. Subsequences can be used to modulate or detect gene expression by introducing into the subsequences compounds which bind, intercalate, cleave and/or crosslink to nucleic acids. Exemplary compounds include acridine, psoralen, phenanthroline, naphthoquinone, daunomycin or chloroethylaminoaryl conjugates.

H. Polynucleotides From a Full-length Enriched cDNA Library Having the Physico-Chemical Property of Selectively Hybridizing to a Polynucleotide of (A)-(G)

As indicated in (h), above, the present invention provides an isolated polynucleotide from a full-length enriched cDNA library having the physico-chemical property of selectively hybridizing to a polynucleotide of paragraphs (A), (B), (C), (D), (E), (F), or (G) as discussed above. Methods of constructing full-length enriched cDNA libraries are known in the art and discussed briefly below. The cDNA library comprises at least 50% to 95% full-length sequences (for example, at least 50%, 60%, 70%, 80%, 90%, or 95% full-length sequences). The cDNA library can be constructed from a variety of tissues from a monocot or dicot at a variety of developmental stages. Exemplary species include maize, wheat, rice, canola, soybean, cotton, sorghum, sunflower, alfalfa, oats, sugar cane, millet, barley, and rice. Methods of selectively hybridizing, under selective hybridization conditions, a polynucleotide from a full-length enriched library to a polynucleotide of the present invention are known to those of ordinary skill in the art. Any number of stringency conditions can be employed to allow for selective hybridization. In optional embodiments, the stringency allows for selective hybridization of sequences having at least 70%, 75%, 80%, 85%, 90%, 95%, or 98% sequence identity over the length of the hybridized region. Full-length enriched cDNA libraries can be normalized to increase the representation of rare sequences.

I. Polynucleotide Products Made by a cDNA Isolation Process

As indicated in (i), above, the present invention provides an isolated polynucleotide made by the process of: 1) providing a full-length enriched nucleic acid library, 2) selectively hybridizing the polynucleotide to a polynucleotide of paragraphs (A), (B), (C), (D), (E), (F), (G), or (H) as discussed above, and thereby isolating the polynucleotide from the nucleic acid library. Full-length enriched nucleic acid libraries are constructed as discussed in paragraph (G) and below. Selective hybridization conditions are as discussed in paragraph (G). Nucleic acid purification procedures are well known in the art. Purification can be conveniently accomplished using solid-phase methods; such methods are well known to those of skill in the art and kits are available from commercial suppliers such as Advanced Biotechnologies (Surrey, UK). For example, a polynucleotide of paragraphs (A)-(H) can be immobilized to a solid support such as a membrane, bead, or particle. See, e.g., U.S. Pat. No. 5,667,976. The polynucleotide product of the present process is selectively hybridized to an immobilized polynucleotide and the solid support is subsequently isolated from non-hybridized polynucleotides by methods including, but not limited to, centrifugation, magnetic separation, filtration, electrophoresis, and the like.

Construction of Nucleic Acids

The isolated nucleic acids of the present invention can be made using (a) standard recombinant methods, (b) synthetic techniques, or (c) combinations thereof. In some embodiments, the polynucleotides of the present invention will be cloned, amplified, or otherwise constructed from a monocot such as corn, rice, or wheat, or a dicot such as soybean.

The nucleic acids may conveniently comprise sequences in addition to a polynucleotide of the present invention. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention. A polynucleotide of the present invention can be attached to a vector, adapter, or linker for cloning and/or expression of a polynucleotide of the present invention. Additional sequences may be added to such cloning and/or expression sequences to optimize their function in cloning and/or expression, to aid in isolation of the polynucleotide, or to improve the introduction of the polynucleotide into a cell. Typically, the length of a nucleic acid of the present invention less the length of its polynucleotide of the present invention is less than 20 kilobase pairs, often less than 15 kb, and frequently less than 10 kb. Use of cloning vectors, expression vectors, adapters, and linkers is well known and extensively described in the art. For a description of various nucleic acids see, for example, Stratagene Cloning Systems, Catalogs 1999 (La Jolla, Calif.); and, Amersham Life Sciences, Inc, Catalog '99 (Arlington Heights, Ill.).

A. Recombinant Methods for Constructing Nucleic Acids

The isolated nucleic acid compositions of this invention, such as RNA, cDNA, genomic DNA, or a hybrid thereof, can be obtained from plant biological sources using any number of cloning methodologies known to those of skill in the art. In some embodiments, oligonucleotide probes which selectively hybridize, under stringent conditions, to the polynucleotides of the present invention are used to identify the desired sequence in a cDNA or genomic DNA library. Isolation of RNA, and construction of cDNA and genomic libraries is well known to those of ordinary skill in the art. See, e.g., Plant Molecular Biology: A Laboratory Manual, Clark, Ed., Springer-Verlag, Berlin (1997); and, Current Protocols in Molecular Biology, Ausubel, et al., Eds., Greene Publishing and Wiley-Interscience, New York (1995).

A1. Full-Length Enriched cDNA Libraries

A number of cDNA synthesis protocols have been described which provide enriched full-length cDNA libraries. Enriched full-length cDNA libraries are constructed to comprise at least 60%, and more preferably at least 70%, 80%, 90% or 95% full-length inserts amongst clones containing inserts. The length of insert in such libraries can be at least 2, 3, 4, 5, 6, 7, 8, 9, 10 or more kilobase pairs. Vectors to accommodate the inserts of these sizes are known in the art and available commercially. See, e.g., Stratagene's lambda ZAP Express (cDNA cloning vector with 0 to 12 kb cloning capacity). An exemplary method of constructing a greater than 95% pure full-length cDNA library is described by Carninci et al., Genomics, 37:327-336 (1996). Other methods for producing full-length libraries are known in the art. See, e.g., Edery et al., Mol. Cell Biol., 15(6):3363-3371 (1995); and, PCT Application WO 96/34981.

A2. Normalized or Subtracted cDNA Libraries

A non-normalized cDNA library represents the mRNA population of the tissue it was made from. Since unique clones are out-numbered by clones derived from highly expressed genes their isolation can be laborious. Normalization of a cDNA library is the process of creating a library in which each clone is more equally represented. Construction of normalized libraries is described in Ko, Nucl. Acids. Res., 18(19):5705-5711 (1990); Patanjali et al., Proc. Natl. Acad. U.S.A., 88:1943-1947 (1991); U.S. Pat. Nos. 5,482,685, 5,482,845, and 5,637,685. In an exemplary method described by Soares et al., normalization resulted in reduction of the abundance of clones from a range of four orders of magnitude to a narrow range of only 1 order of magnitude. Proc. Natl. Acad. Sci. USA, 91:9228-9232 (1994).

Subtracted cDNA libraries are another means to increase the proportion of less abundant cDNA species. In this procedure, cDNA prepared from one pool of mRNA is depleted of sequences present in a second pool of mRNA by hybridization. The cDNA:mRNA hybrids are removed and the remaining un-hybridized cDNA pool is enriched for sequences unique to that pool. See, Foote et al. in, Plant Molecular Biology: A Laboratory Manual, Clark, Ed., Springer-Verlag, Berlin (1997); Kho and Zarbl, Technique, 3(2):58-63 (1991); Sive and St. John, Nucl. Acids Res., 16(22):10937 (1988); Current Protocols in Molecular Biology, Ausubel, et al., Eds., Greene Publishing and Wiley-Interscience, New York (1995); and, Swaroop et al., Nucl. Acids Res., 19)₈):1954 (1991). cDNA subtraction kits are commercially available. See, e.g., PCR-Select (Clontech, Palo Alto, Calif.).

To construct genomic libraries, large segments of genomic DNA are generated by fragmentation, e.g. using restriction endonucleases, and are ligated with vector DNA to form concatemers that can be packaged into the appropriate vector. Methodologies to accomplish these ends, and sequencing methods to verify the sequence of nucleic acids are well known in the art. Examples of appropriate molecular biological techniques and instructions sufficient to direct persons of skill through many construction, cloning, and screening methodologies are found in Sambrook et al., Molecular Cloning: A Laboratory Manual, 2nd Ed., Cold Spring Harbor Laboratory Vols. 1-3 (1989), Methods in Enzymology, Vol. 152: Guide to Molecular Cloning Techniques, Berger and Kimmel, Eds., San Diego: Academic Press, Inc. (1987), Current Protocols in Molecular Biology, Ausubel, et al., Eds., Greene Publishing and Wiley-Interscience, New York (1995); Plant Molecular Biology: A Laboratory Manual, Clark, Ed., Springer-Verlag, Berlin (1997). Kits for construction of genomic libraries are also commercially available.

The cDNA or genomic library can be screened using a probe based upon the sequence of a polynucleotide of the present invention such as those disclosed herein. Probes may be used to hybridize with genomic DNA or cDNA sequences to isolate homologous genes in the same or different plant species. Those of skill in the art will appreciate that various degrees of stringency of hybridization can be employed in the assay; and either the hybridization or the wash medium can be stringent.

The nucleic acids of interest can also be amplified from nucleic acid samples using amplification techniques. For instance, polymerase chain reaction (PCR) technology can be used to amplify the sequences of polynucleotides of the present invention and related genes directly from genomic DNA or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes. The T4 gene 32 protein (Boehringer Mannheim) can be used to improve yield of long PCR products.

PCR-based screening methods have been described. Wilfinger et al. describe a PCR-based method in which the longest cDNA is identified in the first step so that incomplete clones can be eliminated from study. BioTechniques, 22(3):481-486 (1997). Such methods are particularly effective in combination with a full-length cDNA construction methodology, such as that described above.

B. Synthetic Methods for Constructing Nucleic Acids

The isolated nucleic acids of the present invention can also be prepared by direct chemical synthesis using methods such as the phosphotriester method of Narang et al., Meth. Enzymol. 68: 90-99 (1979); the phosphodiester method of Brown et al., Meth. Enzymol. 68: 109-151 (1979); the diethylphosphoramidite method of Beaucage et al., Tetra. Lett. 22: 1859-1862 (1981); the solid phase phosphoramidite triester method described by Beaucage and Caruthers, Tetra. Letts. 22(20): 1859-1862 (1981), e.g., using an automated synthesizer, e.g., as described in Needharn-VanDevanter et al., Nucleic Acids Res., 12: 6159-6168 (1984); and, the solid support method of U.S. Pat. No. 4,458,066. Chemical synthesis generally produces a single stranded oligonucleotide. This may be converted into double stranded DNA by hybridization with a complementary sequence, or by polymerization with a DNA polymerase using the single strand as a template. One of skill will recognize that while chemical synthesis of DNA is best employed for sequences of about 100 bases or less, longer sequences may be obtained by the ligation of shorter sequences.

Recombinant Expression Cassettes

The present invention further provides recombinant expression cassettes comprising a nucleic acid of the present invention. A nucleic acid sequence coding for the desired polypeptide of the present invention, for example a cDNA or a genomic sequence encoding a full length polypeptide of the present invention, can be used to construct a recombinant expression cassette which can be introduced into the desired host cell. A recombinant expression cassette will typically comprise a polynucleotide of the present invention operably linked to transcriptional initiation regulatory sequences which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

A plant promoter fragment can be employed which will direct expression of a polynucleotide of the present invention in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter, and the GRP1-8 promoter.

Alternatively, the plant promoter can direct expression of a polynucleotide of the present invention in a specific tissue or may be otherwise expressed under more precise environmental or developmental control. Such promoters are referred to here as “inducible” promoters. Environmental conditions that may effect transcription by inducible promoters include pathogen attack, anaerobic conditions, or the presence of light. Examples of inducible promoters are the Adhl promoter which is inducible by hypoxia or cold stress, the Hsp70 promoter which is inducible by heat stress, and the PPDK promoter which is inducible by light.

Examples of promoters under developmental control include promoters that initiate transcription only, or preferentially, in certain tissues, such as leaves, roots, fruit, seeds, or flowers. Exemplary promoters include the anther specific promoter 5126 (U.S. Pat. Nos. 5,689,049 and 5,689,051), glob-1 promoter, and gamma-zein promoter. The operation of a promoter may also vary depending on its location in the genome. Thus, an inducible promoter may become fully or partially constitutive in certain locations.

Both heterologous and non-heterologous (i.e., endogenous) promoters can be employed to direct expression of the nucleic acids of the present invention. These promoters can also be used, for example, in recombinant expression cassettes to drive expression of antisense nucleic acids to reduce, increase, or alter concentration and/or composition of the proteins of the present invention in a desired tissue. Thus, in some embodiments, the nucleic acid construct will comprise a promoter, functional in a plant cell, operably linked to a polynucleotide of the present invention. Promoters useful in these embodiments include the endogenous promoters driving expression of a polypeptide of the present invention.

In some embodiments, isolated nucleic acids which serve as promoter or enhancer elements can be introduced in the appropriate position (generally upstream) of a non-heterologous form of a polynucleotide of the present invention so as to up or down regulate expression of a polynucleotide of the present invention. For example, endogenous promoters can be altered in vivo by mutation, deletion, and/or substitution (see, Kmiec, U.S. Pat. No. 5,565,350; Zarling et al., PCT/US93/03868), or isolated promoters can be introduced into a plant cell in the proper orientation and distance from a cognate gene of a polynucleotide of the present invention so as to control the expression of the gene. Gene expression can be modulated under conditions suitable for plant growth so as to alter the total concentration and/or alter the composition of the polypeptides of the present invention in the plant cell. Thus, the present invention provides compositions, and methods for making, heterologous promoters and/or enhancers operably linked to a native, endogenous (i.e., non-heterologous) form of a polynucleotide of the present invention.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added can be derived from, for example, the nopaline synthase or octopine synthase genes, or alternatively from another plant gene, or less preferably from any other eukaryotic gene.

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1: 1183-1200 (1987). Such intron enhancement of gene expression is typically greatest when placed near the 5′ end of the transcription unit. Use of maize introns Adh1-S intron 1, 2, and 6, the Bronze-i intron are known in the art. See generally, The Maize Handbook, Chapter 116, Freeling and Walbot, Eds., Springer, New York (1994). The vector comprising the sequences from a polynucleotide of the present invention will typically comprise a marker gene which confers a selectable phenotype on plant cells. Typical vectors useful for expression of genes in higher plants are well known in the art and include vectors derived from the tumor-inducing (Ti) plasmid of Agrobacterium tumefaciens described by Rogers et al., Meth. in Enzymol., 153:253-277 (1987).

A polynucleotide of the present invention can be expressed in either sense or anti-sense orientation as desired. It will be appreciated that control of gene expression in either sense or anti-sense orientation can have a direct impact on the observable plant characteristics. Antisense technology can be conveniently used to inhibit gene expression in plants. To accomplish this, a nucleic acid segment from the desired gene is cloned and operably linked to a promoter such that the anti-sense strand of RNA will be transcribed. The construct is then transformed into plants and the antisense strand of RNA is produced. In plant cells, it has been shown that antisense RNA inhibits gene expression by preventing the accumulation of mRNA which encodes the enzyme of interest, see, e.g., Sheehy et al., Proc. Nat'l. Acad. Sci. (USA) 85: 8805-8809 (1988); and Hiatt et al., U.S. Pat. No. 4,801,340.

Another method of suppression is sense suppression (i.e., co-supression). Introduction of nucleic acid configured in the sense orientation has been shown to be an effective means by which to block the transcription of target genes. For an example of the use of this method to modulate expression of endogenous genes see, Napoli et al., The Plant Cell 2: 279-289 (1990) and U.S. Pat. No. 5,034,323.

Catalytic RNA molecules or ribozymes can also be used to inhibit expression of plant genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs. The design and use of target RNA-specific ribozymes is described in Haseloffet al., Nature 334: 585-591 (1988).

A variety of cross-linking agents, alkylating agents and radical generating species as pendant groups on polynucleotides of the present invention can be used to bind, label, detect, and/or cleave nucleic acids. For example, Vlassov, V. V., et al., Nucleic Acids Res (1986) 14:4065-4076, describe covalent bonding of a single-stranded DNA fragment with alkylating derivatives of nucleotides complementary to target sequences. A report of similar work by the same group is that by Knorre, D. G., et al., Biochimie (1985) 67:785-789. Iverson and Dervan also showed sequence-specific cleavage of single-stranded DNA mediated by incorporation of a modified nucleotide which was capable of activating cleavage (J Am Chem Soc (1987) 109:1241-1243). Meyer, R. B., et al., J Am Chem Soc (1989) 111:8517-8519, effect covalent crosslinking to a target nucleotide using an alkylating agent complementary to the single-stranded target nucleotide sequence. A photoactivated crosslinking to single-stranded oligonucleotides mediated by psoralen was disclosed by Lee, B. L., et al., Biochemistry (1988) 27:3197-3203. Use of crosslinking in triple-helix forming probes was also disclosed by Home, et al., J Am Chem Soc (1990) 112:2435-2437. Use of N4, N4-ethanocytosine as an alkylating agent to crosslink to single-stranded oligonucleotides has also been described by Webb and Matteucci, J Am Chem Soc (1986) 108:2764-2765; Nucleic Acids Res (1986) 14:7661-7674; Feteritz et al., J. Am. Chem. Soc. 113:4000 (1991). Various compounds to bind, detect, label, and/or cleave nucleic acids are known in the art. See, for example, U.S. Pat. Nos. 5,543,507; 5,672,593; 5,484,908; 5,256,648; and, 5,681941.

Proteins

The isolated proteins of the present invention comprise a polypeptide having at least 10 amino acids from a polypeptide of the present invention (or conservative variants thereof) such as those encoded by any one of the polynucleotides of the present invention as discussed more fully above (e.g., Table 1). The proteins of the present invention, or variants thereof, can comprise any number of contiguous amino acid residues from a polypeptide of the present invention, wherein that number is selected from the group of integers consisting of from 10 to the number of residues in a full-length polypeptide of the present invention. Optionally, this subsequence of contiguous amino acids is at least 15, 20, 25, 30, 35, or 40 amino acids in length, often at least 50, 60, 70, 80, or 90 amino acids in length. Further, the number of such subsequences can be any integer selected from the group consisting of from 1 to 20, such as 2, 3, 4, or 5.

The present invention further provides a protein comprising a polypeptide having a specified sequence identity/similarity with a polypeptide of the present invention. The percentage of sequence identity/similarity is an integer selected from the group consisting of from 50 to 99. Exemplary sequence identity/similarity values include 60%, 65%, 70%, 75%, 80%, 85%, 90%, and 95%. Sequence identity can be determined using, for example, the GAP, CLUSTALW, or BLAST algorithms.

As those of skill in the art will appreciate, the present invention includes, but is not limited to, catalytically active polypeptides of the present invention (i.e., enzymes). Catalytically active polypeptides have a specific activity of at least 20%, 30%, or 40%, and preferably at least 50%, 60%, or 70%, and most preferably at least 80%, 90%, or 95% of that of the native (non-synthetic), endogenous polypeptide. Further, the substrate specificity (k_{cat}/Kₘ) is optionally substantially similar to the native (non-synthetic), endogenous polypeptide. Typically, the Kₘ will be at least 30%, 40%, or 50%, that of the native (non-synthetic), endogenous polypeptide; and more preferably at least 60%, 70%, 80%, or 90%. Methods of assaying and quantifying measures of enzymatic activity and substrate specificity (k_{cat}/Kₘ), are well known to those of skill in the art.

Generally, the proteins of the present invention will, when presented as an immunogen, elicit production of an antibody specifically reactive to a polypeptide of the present invention. Further, the proteins of the present invention will not bind to antisera raised against a polypeptide of the present invention which has been fully immunosorbed with the same polypeptide. Immunoassays for determining binding are well known to those of skill in the art. A preferred immunoassay is a competitive immunoassay. Thus, the proteins of the present invention can be employed as immunogens for constructing antibodies immunoreactive to a protein of the present invention for such exemplary utilities as immunoassays or protein purification techniques.

Expression of Proteins in Host Cells

Using the nucleic acids of the present invention, one may express a protein of the present invention in a recombinantly engineered cell such as bacteria, yeast, insect, mammalian, or preferably plant cells. The cells produce the protein in a non-natural condition (e.g., in quantity, composition, location, and/or time), because they have been genetically altered through human intervention.

It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

In brief summary, the expression of isolated nucleic acids encoding a protein of the present invention will typically be achieved by operably linking, for example, the DNA or cDNA to a promoter (which is either constitutive or regulatable), followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present invention. To obtain high level expression of a cloned gene, it is desirable to construct expression vectors which contain, at the minimum, a strong promoter to direct transcription, a ribosome binding site for translational initiation, and a transcription/translation terminator. One of skill would recognize that modifications can be made to a protein of the present invention without diminishing its biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located purification sequences. Restriction sites or termination codons can also be introduced.

Synthesis of Proteins

The proteins of the present invention can be constructed using non-cellular synthetic methods. Solid phase synthesis of proteins of less than about 50 amino acids in length may be accomplished by attaching the C-terminal amino acid of the sequence to an insoluble support followed by sequential addition of the remaining amino acids in the sequence. Techniques for solid phase synthesis are described by Barany and Merrifield, Solid-Phase Peptide Synthesis, pp. 3-284 in The Peptides: Analysis, Synthesis, Biology. Vol. 2: Special Methods in Peptide Synthesis, Part A.; Merrifield, et al., J. Am. Chem. Soc. 85:2149-2156 (1963), and Stewart et al., Solid Phase Peptide Synthesis, 2nd ed., Pierce Chem. Co., Rockford, Ill. (1984). Proteins of greater length may be synthesized by condensation of the amino and carboxy termini of shorter fragments. Methods of forming peptide bonds by activation of a carboxy terminal end (e.g., by the use of the coupling reagent N,N′-dicycylohexylcarbodiimide) are known to those of skill in the art.

Purification of Proteins

The proteins of the present invention may be purified by standard techniques well known to those of skill in the art. Recombinantly produced proteins of the present invention can be directly expressed or expressed as a fusion protein. The recombinant protein is purified by a combination of cell lysis (e.g., sonication, French press) and affinity chromatography. For fusion products, subsequent digestion of the fusion protein with an appropriate proteolytic enzyme releases the desired recombinant protein.

The proteins of this invention, recombinant or synthetic, may be purified to substantial purity by standard techniques well known in the art, including detergent solubilization, selective precipitation with such substances as ammonium sulfate, column chromatography, immunopurification methods, and others. See, for instance, R. Scopes, Protein Purification: Principles and Practice, Springer-Verlag: New York (1982); Deutscher, Guide to Protein Purification, Academic Press (1990). For example, antibodies may be raised to the proteins as described herein. Purification from E. coli can be achieved following procedures described in U.S. Pat. No. 4,511,503. The protein may then be isolated from cells expressing the protein and further purified by standard protein chemistry techniques as described herein. Detection of the expressed protein is achieved by methods known in the art and include, for example, radioimmunoassays, Western blotting techniques or immunoprecipitation.

Introduction of Nucleic Acids into Host Cells

The method of introducing a nucleic acid of the present invention into a host cell is not critical to the instant invention. Transformation or transfection methods are conveniently used. Accordingly, a wide variety of methods have been developed to insert a DNA sequence into the genome of a host cell to obtain the transcription and/or translation of the sequence to effect phenotypic changes in the organism. Thus, any method which provides for effective introduction of a nucleic acid may be employed.

A. Plant Transformation

A nucleic acid comprising a polynucleotide of the present invention is optionally introduced into a plant. Generally, the polynucleotide will first be incorporated into a recombinant expression cassette or vector. Isolated nucleic acid acids of the present invention can be introduced into plants according to techniques known in the art. Techniques for transforming a wide variety of higher plant species are well known and described in the technical, scientific, and patent literature. See, for example, Weising et al., Ann. Rev. Genet. 22:421-477 (1988). For example, the DNA construct may be introduced directly into the genomic DNA of the plant cell using techniques such as electroporation, polyethylene glycol (PEG), poration, particle bombardment, silicon fiber delivery, or microinjection of plant cell protoplasts or embryogenic callus. See, e.g., Tomes, et al., Direct DNA Transfer into Intact Plant Cells Via Microprojectile Bombardment. pp. 197-213 in Plant Cell, Tissue and Organ Culture, Fundamental Methods. eds. O. L. Gamborg and G. C. Phillips. Springer-Verlag Berlin Heidelberg New York, 1995; see, U.S. Pat. No. 5,990,387. The introduction of DNA constructs using PEG precipitation is described in Paszkowski et al., Embo J. 3:2717-2722 (1984). Electroporation techniques are described in Fromm et al., Proc. Natl. Acad. Sci. (USA) 82:5824 (1985). Ballistic transformation techniques are described in Klein et al., Nature 327:70-73 (1987) and in U.S. Pat. No. 4,945,050.

Agrobacterium tumefaciens-mediated transformation techniques are well described in the scientific literature. See, for example Horsch et al., Science 233: 496-498 (1984); Fraley et al., Proc. Natl. Acad. Sci. (USA) 80: 4803 (1983); and, Plant Molecular Biology: A Laboratory Manual, Chapter 8, Clark, Ed., Springer-Verlag, Berlin (1997). The DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. See, U.S. Pat. No. 5,591,616. Although Agrobacterium is useful primarily in dicots, certain monocots can be transformed by Agrobacterium. For instance, Agrobacterium transformation of maize is described in U.S. Pat. No. 5,550,318.

Other methods of transfection or transformation include (1) Agrobacterium rhizogenes-mediated transformation (see, e.g., Lichtenstein and Fuller In: Genetic Engineering, vol. 6, P W J Rigby, Ed., London, Academic Press, 1987; and Lichtenstein, C. P., and Draper, J,. In: DNA Cloning, Vol. 11, D. M. Glover, Ed., Oxford, IR1 Press, 1985), Application PCT/US87/02512 (WO 88/02405 published Apr. 7, 1988) describes the use of A. rhizogenes strain A4 and its R1 plasmid along with A. tumefaciens vectors pARC8 or pARC16, (2) liposome-mediated DNA uptake (see, e.g., Freeman et al., Plant Cell Physiol. 25: 1353 (1984)), and (3) the vortexing method (see, e.g., Kindle, Proc. Natl. Acad. Sci., (USA) 87: 1228 (1990).

DNA can also be introduced into plants by direct DNA transfer into pollen as described by Zhou et al., Methods in Enzymology, 101:433 (1983); D. Hess, Intern Rev. Cytol., 107:367 (1987); Luo et al., Plant Mol. Biol. Reporter, 6:165 (1988). Expression of polypeptide coding genes can be obtained by injection of the DNA into reproductive organs of a plant as described by Pena et al., Nature, 325:274 (1987). DNA can also be injected directly into the cells of immature embryos and the rehydration of desiccated embryos as described by Neuhaus et al., Theor. Appl. Genet., 75:30 (1987); and Benbrook et al., in Proceedings Bio Expo 1986, Butterworth, Stoneham, Mass., pp. 27-54 (1986). A variety of plant viruses that can be employed as vectors are known in the art and include cauliflower mosaic virus (CaMV), geminivirus, brome mosaic virus, and tobacco mosaic virus.

B. Transfection of Prokaryotes, Lower Eukaryotes, and Animal Cells

Animal and lower eukaryotic (e.g., yeast) host cells are competent or rendered competent for transfection by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextran, electroporation, biolistics, and micro-injection of the DNA directly into the cells. The transfected cells are cultured by means well known in the art. Kuchler, R. J., Biochemical Methods in Cell Culture and Virology, Dowden, Hutchinson and Ross, Inc. (1977).

Transgenic Plant Regeneration

Plant cells which directly result or are derived from the nucleic acid introduction techniques can be cultured to regenerate a whole plant that possesses the introduced genotype. Such regeneration techniques often rely on manipulation of certain phytohormones in a tissue culture growth medium. Plants cells can be regenerated, e.g., from single cells, callus tissue or leaf discs according to standard plant tissue culture techniques. It is well known in the art that various cells, tissues, and organs from almost any plant can be successfully cultured to regenerate an entire plant. Plant regeneration from cultured protoplasts is described in Evans et al., Protoplasts Isolation and Culture, Handbook of Plant Cell Culture, Macmillan Publishing Company, New York, pp. 124-176 (1983); and Binding, Regeneration of Plants, Plant Protoplasts, CRC Press, Boca Raton, pp. 21-73 (1985).

The regeneration of plants from either single plant protoplasts or various explants is well known in the art. See, for example, Methods for Plant Molecular Biology, A. Weissbach and H. Weissbach, eds., Academic Press, Inc., San Diego, Calif. (1988). This regeneration and growth process includes the steps of selection of transformant cells and shoots, and rooting the transformant shoots and growth of the plantlets in soil. For maize cell culture and regeneration see generally, The Maize Handbook, Freeling and Walbot, Eds., Springer, NY (1994); Corn and Corn Improvement, 3^{rd} edition, Sprague and Dudley Eds., American Society of Agronomy, Madison, Wis. (1988). For transformation and regeneration of maize see, Gordon-Kamm et al., The Plant Cell, 2:603-618 (1990).

The regeneration of plants containing the polynucleotide of the present invention and introduced by Agrobacterium from leaf explants can be achieved as described by Horsch et al., Science, 227:1229-1231 (1985). In this procedure, transformants are grown in the presence of a selection agent and in a medium that induces the regeneration of shoots in the plant species being transformed as described by Fraley et al., Proc. Natl. Acad. Sci. (U.S.A.), 80:4803 (1983). This procedure typically produces shoots within two to four weeks and these transformant shoots are then transferred to an appropriate root-inducing medium containing the selective agent and an antibiotic to prevent bacterial growth. Transgenic plants of the present invention may be fertile or sterile.

One of skill will recognize that after the recombinant expression cassette is stably incorporated in transgenic plants and confirmed to be operable, it can be introduced into other plants by sexual crossing. Any of a number of standard breeding techniques can be used, depending upon the species to be crossed. In vegetatively propagated crops, mature transgenic plants can be propagated by the taking of cuttings or by tissue culture techniques to produce multiple identical plants. Selection of desirable transgenics is made and new varieties are obtained and propagated vegetatively for commercial use. In seed propagated crops, mature transgenic plants can be self crossed to produce a homozygous inbred plant. The inbred plant produces seed containing the newly introduced heterologous nucleic acid. These seeds can be grown to produce plants that would produce the selected phenotype. Parts obtained from the regenerated plant, such as flowers, seeds, leaves, branches, fruit, and the like are included in the invention, provided that these parts comprise cells comprising the isolated nucleic acid of the present invention. Progeny and variants, and mutants of the regenerated plants are also included within the scope of the invention, provided that these parts comprise the introduced nucleic acid sequences.

Transgenic plants expressing a polynucleotide of the present invention can be screened for transmission of the nucleic acid of the present invention by, for example, standard immunoblot and DNA detection techniques. Expression at the RNA level can be determined initially to identify and quantitate expression-positive plants. Standard techniques for RNA analysis can be employed and include PCR amplification assays using oligonucleotide primers designed to amplify only the heterologous RNA templates and solution hybridization assays using heterologous nucleic acid-specific probes. The RNA-positive plants can then analyzed for protein expression by Western immunoblot analysis using the specifically reactive antibodies of the present invention. In addition, in situ hybridization and immunocytochemistry according to standard protocols can be done using heterologous nucleic acid specific polynucleotide probes and antibodies, respectively, to localize sites of expression within transgenic tissue. Generally, a number of transgenic lines are usually screened for the incorporated nucleic acid to identify and select plants with the most appropriate expression profiles.

A preferred embodiment is a transgenic plant that is homozygous for the added heterologous nucleic acid; i.e., a transgenic plant that contains two added nucleic acid sequences, one gene at the same locus on each chromosome of a chromosome pair. A homozygous transgenic plant can be obtained by sexually mating (selfing) a heterozygous transgenic plant that contains a single added heterologous nucleic acid, germinating some of the seed produced and analyzing the resulting plants produced for altered expression of a polynucleotide of the present invention relative to a control plant (i.e., native, non-transgenic). Back-crossing to a parental plant and out-crossing with a non-transgenic plant are also contemplated.

Modulating Polypeptide Levels and/or Composition

The present invention further provides a method for modulating (i.e., increasing or decreasing) the concentration or ratio of the polypeptides of the present invention in a plant or part thereof. Modulation can be effected by increasing or decreasing the concentration and/or the ratio of the polypeptides of the present invention in a plant. The method comprises introducing into a plant cell a recombinant expression cassette comprising a polynucleotide of the present invention as described above to obtain a transgenic plant cell, culturing the transgenic plant cell under transgenic plant cell growing conditions, and inducing or repressing expression of a polynucleotide of the present invention in the transgenic plant for a time sufficient to modulate concentration and/or the ratios of the polypeptides in the transgenic plant or plant part.

In some embodiments, the concentration and/or ratios of polypeptides of the present invention in a plant may be modulated by altering, in vivo or in vitro, the promoter of a gene to up- or down-regulate gene expression. In some embodiments, the coding regions of native genes of the present invention can be altered via substitution, addition, insertion, or deletion to decrease activity of the encoded enzyme. See, e.g., Kmiec, U.S. Pat. No. 5,565,350; Zarling et al., PCT/US93/03868. And in some embodiments, an isolated nucleic acid (e.g., a vector) comprising a promoter sequence is transfected into a plant cell. Subsequently, a plant cell comprising the promoter operably linked to a polynucleotide of the present invention is selected for by means known to those of skill in the art such as, but not limited to, Southern blot, DNA sequencing, or PCR analysis using primers specific to the promoter and to the gene and detecting amplicons produced therefrom. A plant or plant part altered or modified by the foregoing embodiments is grown under plant forming conditions for a time sufficient to modulate the concentration and/or ratios of polypeptides of the present invention in the plant. Plant forming conditions are well known in the art and discussed briefly, supra.

In general, concentration or the ratios of the polypeptides is increased or decreased by at least 5%, 10%, 20%, 30%, 40%, 50%, 60%, 70%, 80%, or 90% relative to a native control plant, plant part, or cell lacking the aforementioned recombinant expression cassette. Modulation in the present invention may occur during and/or subsequent to growth of the plant to the desired stage of development. Modulating nucleic acid expression temporally and/or in particular tissues can be controlled by employing the appropriate promoter operably linked to a polynucleotide of the present invention in, for example, sense or antisense orientation as discussed in greater detail, supra. Induction of expression of a polynucleotide of the present invention can also be controlled by exogenous administration of an effective amount of inducing compound. Inducible promoters and inducing compounds which activate expression from these promoters are well known in the art. In preferred embodiments, the polypeptides of the present invention are modulated in monocots, particularly maize.

UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, Nucleic Acids Res. 15:8125 (1987)) and the 7-methylguanosine cap structure (Drummond et al., Nucleic Acids Res. 13:7375 (1985)). Negative elements include stable intramolecular 5′ UTR stem-loop structures (Muesing et al., Cell 48:691 (1987)) and AUG sequences or short open reading frames preceded by an appropriate AUG in the 5′ UTR (Kozak, supra, Rao et al., Mol. and Cell. Biol. 8:284 (1988)). Accordingly, the present invention provides 5′ and/or 3′ untranslated regions for modulation of translation of heterologous coding sequences.

Further, the polypeptide-encoding segments of the polynucleotides of the present invention can be modified to alter codon usage. Altered codon usage can be employed to alter translational efficiency and/or to optimize the coding sequence for expression in a desired host such as to optimize the codon usage in a heterologous sequence for expression in maize. Codon usage in the coding regions of the polynucleotides of the present invention can be analyzed statistically using commercially available software packages such as “Codon Preference” available from the University of Wisconsin Genetics Computer Group (see Devereaux et al., Nucleic Acids Res. 12:387-395 (1984)) or MacVector 4.1 (Eastman Kodak Co., New Haven, Conn.). Thus, the present invention provides a codon usage frequency characteristic of the coding region of at least one of the polynucleotides of the present invention. The number of polynucleotides that can be used to determine a codon usage frequency can be any integer from 1 to the number of polynucleotides of the present invention as provided herein. Optionally, the polynucleotides will be full-length sequences. An exemplary number of sequences for statistical analysis can be at least 1, 5, 10, 20, 50, or 100.

Sequence Shuffling

The present invention provides methods for sequence shuffling using polynucleotides of the present invention, and compositions resulting therefrom. Sequence shuffling is described in PCT publication No. WO 97/20078. See also, Zhang, J.-H., et al. Proc. Natl. Acad. Sci. USA 94:4504-4509 (1997). Generally, sequence shuffling provides a means for generating libraries of polynucleotides having a desired characteristic which can be selected or screened for. Libraries of recombinant polynucleotides are generated from a population of related sequence polynucleotides which comprise sequence regions which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation, or other expression property of a gene or transgene, a replicative element, a protein-binding element, or the like, such as any feature which confers a selectable or detectable property. In some embodiments, the selected characteristic will be a decreased Kₘ and/or increased K_{cat} over the wild-type protein as provided herein. In other embodiments, a protein or polynucleotide generated from sequence shuffling will have a ligand binding affinity greater than the non-shuffled wild-type polynucleotide. The increase in such properties can be at least 110%, 120%, 130%, 140% or at least 150% of the wild-type value.

Generic and Consensus Sequences

Polynucleotides and polypeptides of the present invention further include those having: (a) a generic sequence of at least two homologous polynucleotides or polypeptides, respectively, of the present invention; and, (b) a consensus sequence of at least three homologous polynucleotides or polypeptides, respectively, of the present invention. The generic sequence of the present invention comprises each species of polypeptide or polynucleotide embraced by the generic polypeptide or polynucleotide sequence, respectively. The individual species encompassed by a polynucleotide having an amino acid or nucleic acid consensus sequence can be used to generate antibodies or produce nucleic acid probes or primers to screen for homologs in other species, genera, families, orders, classes, phyla, or kingdoms. For example, a polynucleotide having a consensus sequence from a gene family of Zea mays can be used to generate antibody or nucleic acid probes or primers to other Gramineae species such as wheat, rice, or sorghum. Alternatively, a polynucleotide having a consensus sequence generated from orthologous genes can be used to identify or isolate orthologs of other taxa. Typically, a polynucleotide having a consensus sequence will be at least 9, 10, 15, 20, 25, 30, or 40 amino acids in length, or 20, 30, 40, 50, 100, or 150 nucleotides in length. As those of skill in the art are aware, a conservative amino acid substitution can be used for amino acids which differ amongst aligned sequence but are from the same conservative substitution group as discussed above. Optionally, no more than 1 or 2 conservative amino acids are substituted for each 10 amino acid length of consensus sequence.

Similar sequences used for generation of a consensus or generic sequence include any number and combination of allelic variants of the same gene, orthologous, or paralogous sequences as provided herein. Optionally, similar sequences used in generating a consensus or generic sequence are identified using the BLAST algorithm's smallest sum probability (P(N)). Various suppliers of sequence-analysis software are listed in chapter 7 of Current Protocols in Molecular Biology, F. M. Ausubel et al., Eds., Current Protocols, a joint venture between Greene Publishing Associates, Inc. and John Wiley & Sons, Inc. (Supplement 30). A polynucleotide sequence is considered similar to a reference sequence if the smallest sum probability in a comparison of the test nucleic acid to the reference nucleic acid is less than about 0.1, more preferably less than about 0.01, or 0.001, and most preferably less than about 0.0001, or 0.00001. Similar polynucleotides can be aligned and a consensus or generic sequence generated using multiple sequence alignment software available from a number of commercial suppliers such as the Genetics Computer Group's (Madison, Wis.) PILEUP software, Vector NTI's (North Bethesda, Md.) ALIGNX, or Genecode's (Ann Arbor, Mich.) SEQUENCHER. Conveniently, default parameters of such software can be used to generate consensus or generic sequences.

Machine Applications

The present invention provides machines, data structures, and processes for modeling or analyzing the polynucleotides and polypeptides of the present invention.

A. Machines: Data, Data Structures, Processes, and Functions

The present invention provides a machine having a memory comprising: 1) data representing a sequence of a polynucleotide or polypeptide of the present invention, 2) a data structure which reflects the underlying organization and structure of the data and facilitates program access to data elements corresponding to logical sub-components of the sequence, 3) processes for effecting the use, analysis, or modeling of the sequence, and 4) optionally, a function or utility for the polynucleotide or polypeptide. Thus, the present invention provides a memory for storing data that can be accessed by a computer programmed to implement a process for effecting the use, analyses, or modeling of a sequence of a polynucleotide, with the memory comprising data representing the sequence of a polynucleotide of the present invention.

The machine of the present invention is typically a digital computer. The term “computer” includes one or several desktop or portable computers, computer workstations, servers (including intranet or internet servers), mainframes, and any integrated system comprising any of the above irrespective of whether the processing, memory, input, or output of the computer is remote or local, as well as any networking interconnecting the modules of the computer. The term “computer” is exclusive of computers of the United States Patent and Trademark Office or the European Patent Office when data representing the sequence of polypeptides or polynucleotides of the present invention is used for patentability searches.

The present invention contemplates providing, as data, a sequence of a polynucleotide of the present invention embodied in a computer readable medium. As those of skill in the art will be aware, the form of memory of a machine of the present invention, or the particular embodiment of the computer readable medium, are not critical elements of the invention and can take a variety of forms. The memory of such a machine includes, but is not limited to, ROM, or RAM, or computer readable media such as, but not limited to, magnetic media such as computer disks or hard drives, or media such as CD-ROMs, DVDs, and the like.

The present invention further contemplates providing a data structure that is also contained in memory. The data structure may be defined by the computer programs that define the processes (see below) or it may be defined by the programming of separate data storage and retrieval programs subroutines, or systems. Thus, the present invention provides a memory for storing a data structure that can be accessed by a computer programmed to implement a process for effecting the use, analysis, or modeling of a sequence of a polynucleotide. The memory comprises data representing a polynucleotide having the sequence of a polynucleotide of the present invention. The data is stored within memory. Further, a data structure, stored within memory, is associated with the data reflecting the underlying organization and structure of the data to facilitate program access to data elements corresponding to logical sub-components of the sequence. The data structure enables the polynucleotide to be identified and manipulated by such programs.

In a further embodiment, the present invention provides a data structure that contains data representing a sequence of a polynucleotide of the present invention stored within a computer readable medium. The data structure is organized to reflect the logical structuring of the sequence, so that the sequence is easily analyzed by software programs capable of accessing the data structure. In particular, the data structures of the present invention organize the reference sequences of the present invention in a manner which allows software tools to perform a wide variety of analyses using logical elements and sub-elements of each sequence.

An example of such a data structure resembles a layered hash table, where in one dimension the base content of the sequence is represented by a string of elements A, T, C, G and N. The direction from the 5′ end to the 3′ end is reflected by the order from the position 0 to the position of the length of the string minus one. Such a string, corresponding to a nucleotide sequence of interest, has a certain number of substrings, each of which is delimited by the string position of its 5′ end and the string position of its 3′ end within the parent string. In a second dimension, each substring is associated with or pointed to one or multiple attribute fields. Such attribute fields contain annotations to the region on the nucleotide sequence represented by the substring.

For example, a sequence under investigation is 520 bases long and represented by a string named SeqTarget. There is a minor groove in the 5′ upstream non-coding region from position 12 to 38, which is identified as a binding site for an enhancer protein HM-A, which in turn will increase the transcription of the gene represented by SeqTarget. Here, the substring is represented as (12, 38) and has the following attributes: [upstream uncoded], [minor groove], [HM-A binding] and [increase transcription upon binding by HM-A]. Similarly, other types of information can be stored and structured in this manner, such as information related to the whole sequence, e.g., whether the sequence is a full length viral gene, a mammalian house keeping gene or an EST from clone X, information related to the 3′ down stream non-coding region, e.g., hair pin structure, and information related to various domains of the coding region, e.g., Zinc finger.

This data structure is an open structure and is robust enough to accommodate newly generated data and acquired knowledge. Such a structure is also a flexible structure. It can be trimmed down to a 1-D string to facilitate data mining and analysis steps, such as clustering, repeat-masking, and HMM analysis. Meanwhile, such a data structure also can extend the associated attributes into multiple dimensions. Pointers can be established among the dimensioned attributes when needed to facilitate data management and processing in a comprehensive genomics knowledgebase. Furthermore, such a data structure is object-oriented. Polymorphism can be represented by a family or class of sequence objects, each of which has an internal structure as discussed above. The common traits are abstracted and assigned to the parent object, whereas each child object represents a specific variant of the family or class. Such a data structure allows data to be efficiently retrieved, updated and integrated by the software applications associated with the sequence database and/or knowledgebase.

The present invention contemplates providing processes for effecting analysis and modeling, which are described in the following section.

Optionally, the present invention further contemplates that the machine of the present invention will embody in some manner a utility or function for the polynucleotide or polypeptide of the present invention. The function or utility of the polynucleotide or polypeptide can be a function or utility for the sequence data, per se, or of the tangible material. Exemplary function or utilities include the name (per International Union of Biochemistry and Molecular Biology rules of nomenclature) or function of the enzyme or protein represented by the polynucleotide or polypeptide of the present invention; the metabolic pathway of the protein represented by the polynucleotide or polypeptide of the present invention; the substrate or product or structural role of the protein represented by the polynucleotide or polypeptide of the present invention; or, the phenotype (e.g., an agronomic or pharmacological trait) affected by modulating expression or activity of the protein represented by the polynucleotide or polypeptide of the present invention.

B. Computer Analysis and Modeling

The present invention provides a process of modeling and analyzing data representative of a polynucleotide or polypeptide sequence of the present invention. The process comprises entering sequence data of a polynucleotide or polypeptide of the present invention into a machine having a hardware or software sequence modeling and analysis system, developing data structures to facilitate access to the sequence data, manipulating the data to model or analyze the structure or activity of the polynucleotide or polypeptide, and displaying the results of the modeling or analysis. Thus, the present invention provides a process for effecting the use, analysis, or modeling of a polynucleotide sequence or its derived peptide sequence through use of a computer having a memory. The process comprises 1) placing into memory the data representing a polynucleotide having the sequence of a polynucleotide of the present invention, developing within the memory a data structure associated with the data and reflecting the underlying organization and structure of the data to facilitate program access to data elements corresponding to logical sub-components of the sequence, 2) programming the computer with a program containing instructions sufficient to implement the process for effecting the use, analysis, or modeling of the polynucleotide sequence or the peptide sequence, 3) executing the program on the computer while granting the program access to the data and to the data structure within the memory, and 4) outputting a set of results of said process.

A variety of modeling and analytic tools are well known in the art and available commercially. Included amongst the modeling/analysis tools are methods to: 1) recognize overlapping sequences (e.g., from a sequencing project) with a polynucleotide of the present invention and create an alignment called a “contig”; 2) identify restriction enzyme sites of a polynucleotide of the present invention; 3) identify the products of a T1 ribonuclease digestion of a polynucleotide of the present invention; 4) identify PCR primers with minimal self-complementarity; 5) compute pairwise distances between sequences in an alignment, reconstruct phylogentic trees using distance methods, and calculate the degree of divergence of two protein coding regions; 6) identify patterns such as coding regions, terminators, repeats, and other consensus patterns in polynucleotides of the present invention; 7) identify RNA secondary structure; 8) identify sequence motifs, isoelectric point, secondary structure, hydrophobicity, and antigenicity in polypeptides of the present invention; 9) translate polynucleotides of the present invention and backtranslate polypeptides of the present invention; and 10) compare two protein or nucleic acid sequences and identifying points of similarity or dissimilarity between them.

The processes for effecting analysis and modeling can be produced independently or obtained from commercial suppliers. Exemplary analysis and modeling tools are provided in products such as InforMax's (Bethesda, Md.) Vector NTI Suite (Version 5.5), Intelligenetics' (Mountain View, Calif.) PC/Gene program, and Genetics Computer Group's (Madison, Wis.) Wisconsin Package (Version 10.0); these tools, and the functions they perform, (as provided and disclosed by the programs and accompanying literature) are incorporated herein by reference and are described in more detail in section C which follows.

Thus, in a further embodiment, the present invention provides a machine-readable media containing a computer program and data, comprising a program stored on the media containing instructions sufficient to implement a process for effecting the use, analysis, or modeling of a representation of a polynucleotide or peptide sequence. The data stored on the media represents a sequence of a polynucleotide having the sequence of a polynucleotide of the present invention. The media also includes a data structure reflecting the underlying organization and structure of the data to facilitate program access to data elements corresponding to logical sub-components of the sequence, the data structure being inherent in the program and in the way in which the program organizes and accesses the data.

C. Homology Searches

As an example of such a comparative analysis, the present invention provides a process of identifying a candidate homologue (i.e., an ortholog or paralog) of a polynucleotide or polypeptide of the present invention. The process comprises entering sequence data of a polynucleotide or polypeptide of the present invention into a machine having a hardware or software sequence analysis system, developing data structures to facilitate access to the sequence data, manipulating the data to analyze the structure the polynucleotide or polypeptide, and displaying the results of the analysis. A candidate homologue has statistically significant probability of having the same biological function (e.g., catalyzes the same reaction, binds to homologous proteins/nucleic acids, has a similar structural role) as the reference sequence to which it is compared. Accordingly, the polynucleotides and polypeptides of the present invention have utility in identifying homologs in animals or other plant species, particularly those in the family Gramineae such as, but not limited to, sorghum, wheat, or rice.

The process of the present invention comprises obtaining data representing a polynucleotide or polypeptide test sequence. Test sequences can be obtained from a nucleic acid of an animal or plant. Test sequences can be obtained directly or indirectly from sequence databases including, but not limited to, those such as: GenBank, EMBL, GenSeq, SWISS-PROT, or those available on-line via the UK Human Genome Mapping Project (HGMP) GenomeWeb. In some embodiments the test sequence is obtained from a plant species other than maize whose function is uncertain but will be compared to the test sequence to determine sequence similarity or sequence identity. The test sequence data is entered into a machine, such as a computer, containing: i) data representing a reference sequence and, ii) a hardware or software sequence comparison system to compare the reference and test sequence for sequence similarity or identity.

Exemplary sequence comparison systems are provided for in sequence analysis software such as those provided by the Genetics Computer Group (Madison, Wis.) or InforMax (Bethesda, Md.), or Intelligenetics (Mountain View, Calif.). Optionally, sequence comparison is established using the BLAST or GAP suite of programs. Generally, a smallest sum probability value (P(N)) of less than 0.1, or alternatively, less than 0.01, 0.001, 0.0001, or 0.00001 using the BLAST 2.0 suite of algorithms under default parameters identifies the test sequence as a candidate homologue (i.e., an allele, ortholog, or paralog) of the reference sequence. Those of skill in the art will recognize that a candidate homologue has an increased statistical probability of having the same or similar function as the gene/protein represented by the test sequence.

The reference sequence can be the sequence of a polypeptide or a polynucleotide of the present invention. The reference or test sequence is each optionally at least 25 amino acids or at least 100 nucleotides in length. The length of the reference or test sequences can be the length of the polynucleotide or polypeptide described, respectively, above in the sections entitled “Nucleic Acids” (particularly section (g)), and “Proteins”. As those of skill in the art are aware, the greater the sequence identity/similarity between a reference sequence of known function and a test sequence, the greater the probability that the test sequence will have the same or similar function as the reference sequence. The results of the comparison between the test and reference sequences are outputted (e.g., displayed, printed, recorded) via any one of a number of output devices and/or media (e.g., computer monitor, hard copy, or computer readable medium).

Detection of Nucleic Acids

The present invention further provides methods for detecting a polynucleotide of the present invention in a nucleic acid sample suspected of containing a polynucleotide of the present invention, such as a plant cell lysate, particularly a lysate of maize. In some embodiments, a cognate gene of a polynucleotide of the present invention or substantial portion thereof can be amplified prior to the step of contacting the nucleic acid sample with a polynucleotide of the present invention. The nucleic acid sample is contacted with the polynucleotide to form a hybridization complex. The polynucleotide hybridizes under stringent conditions to a gene encoding a polypeptide of the present invention. Formation of the hybridization complex is used to detect a gene encoding a polypeptide of the present invention in the nucleic acid sample. Those of skill will appreciate that an isolated nucleic acid comprising a polynucleotide of the present invention should lack cross-hybridizing sequences in common with non-target genes that would yield a false positive result. Detection of the hybridization complex can be achieved using any number of well known methods. For example, the nucleic acid sample, or a substantial portion thereof, may be assayed by hybridization formats including but not limited to, solution phase, solid phase, mixed phase, or in situ hybridization assays.

Detectable labels suitable for use in the present invention include any composition detectable by spectroscopic, radioisotopic, photochemical, biochemical, immunochemical, electrical, optical or chemical means. Useful labels in the present invention include biotin for staining with labeled streptavidin conjugate, magnetic beads, fluorescent dyes, radiolabels, enzymes, and colorimetric labels. Other labels include ligands which bind to antibodies labeled with fluorophores, chemiluminescent agents, and enzymes. Labeling the nucleic acids of the present invention is readily achieved such as by the use of labeled PCR primers.

Although the present invention has been described in some detail by way of illustration and example for purposes of clarity of understanding, it will be obvious that certain changes and modifications may be practiced within the scope of the appended claims.

### EXAMPLE 1

### The Construction of a cDNA Library

Total RNA can be isolated from maize tissues with TRIzol Reagent (Life Technology Inc. Gaithersburg, Md.) using a modification of the guanidine isothiocyanate/acid-phenol procedure described by Chomczynski and Sacchi (Chomczynski, P., and Sacchi, N. Anal. Biochem. 162, 156 (1987)). In brief, plant tissue samples are pulverized in liquid nitrogen before the addition of the TRIzol Reagent, and then further homogenized with a mortar and pestle. Addition of chloroform followed by centrifugation is conducted for separation of an aqueous phase and an organic phase. The total RNA is recovered by precipitation with isopropyl alcohol from the aqueous phase.

The selection of poly(A)+ RNA from total RNA can be performed using PolyATact system (Promega Corporation. Madison, Wis.). Biotinylated oligo(dT) primers are used to hybridize to the 3′ poly(A) tails on mRNA. The hybrids are captured using streptavidin coupled to paramagnetic particles and a magnetic separation stand. The mRNA is then washed at high stringency conditions and eluted by RNase-free deionized water.

cDNA synthesis and construction of unidirectional cDNA libraries can be accomplished using the SuperScript Plasmid System (Life Technology Inc. Gaithersburg, Md.). The first strand of cDNA is synthesized by priming an oligo(dT) primer containing a Not I site. The reaction is catalyzed by SuperScript Reverse Transcriptase II at 45° C. The second strand of cDNA is labeled with alpha-³²P-dCTP and a portion of the reaction analyzed by agarose gel electrophoresis to determine cDNA sizes. cDNA molecules smaller than 500 base pairs and unligated adapters are removed by Sephacryl-S400 chromatography. The selected cDNA molecules are ligated into pSPORT1 vector in between of Not I and Sal I sites.

Alternatively, cDNA libraries can be prepared by any one of many methods available. For example, the cDNAs may be introduced into plasmid vectors by first preparing the cDNA libraries in Uni-ZAP™ XR vectors according to the manufacturer's protocol (Stratagene Cloning Systems, La Jolla, Calif.). The Uni-ZAP™ XR libraries are converted into plasmid libraries according to the protocol provided by Stratagene. Upon conversion, cDNA inserts will be contained in the plasmid vector pBluescript. In addition, the cDNAs may be introduced directly into precut Bluescript II SK(+) vectors (Stratagene) using T4 DNA ligase (New England Biolabs), followed by transfection into DH10B cells according to the manufacturer's protocol (GIBCO BRL Products). Once the cDNA inserts are in plasmid vectors, plasmid DNAs are prepared from randomly picked bacterial colonies containing recombinant pBluescript plasmids, or the insert cDNA sequences are amplified via polymerase chain reaction using primers specific for vector sequences flanking the inserted cDNA sequences. Amplified insert DNAs or plasmid DNAs are sequenced in dye-primer sequencing reactions to generate partial cDNA sequences (expressed sequence tags or “ESTs”; see Adams et al., (1991) Science 252:1651-1656). The resulting ESTs are analyzed using a Perkin Elmer Model 377 fluorescent sequencer.

### EXAMPLE 2

### The Construction of a Full-Length Enriched cDNA Library

An enriched full-length cDNA library can be constructed using one of two variations of the method of Carninci et al. Genomics 37:327-336, 1996. These variations are based on chemical introduction of a biotin group into the diol residue of the 5′ cap structure of eukaryotic mRNA to select full-length first strand cDNA. The selection occurs by trapping the biotin residue at the cap sites using streptavidin-coated magnetic beads followed by RNase I treatment to eliminate incompletely synthesized cDNAs. Second strand cDNA is synthesized using established procedures such as those provided in Life Technologies' (Rockville, Md.) “SuperScript Plasmid System for cDNA Synthesis and Plasmid Cloning” kit. Libraries made by this method have been shown to contain 50% to 70% full-length cDNAs.

The first strand synthesis methods are detailed below. An asterisk denotes that the reagent was obtained from Life Technologies, Inc.

A. First Strand cDNA Synthesis Method I (with Trehalose)

| | | |
|---|---|---|
| mRNA (10 ug) | 25 | μL |
| *Not I primer (5 ug) | 10 | μL |
| *5x 1^{st} strand buffer | 43 | μL |
| *0.1 m DTT | 20 | μL |
| *dNTP mix 10 mm | 10 | μL |
| BSA 10 ug/μL | 1 | μL |
| Trehalose (saturated) | 59.2 | μL |
| RNase inhibitor (Promega) | 1.8 | μL |
| *Superscript II RT 200 u/μL | 20 | μL |
| 100% glycerol | 18 | μL |
| Water | 7 | μL |

The mRNA and Not I primer are mixed and denatured at 65° C. for 10 min. They are then chilled on ice and other components added to the tube. Incubation is at 45° C. for 2 min. Twenty microliters of RT (reverse transcriptase) is added to the reaction and start program on the thermocycler (MJ Research, Waltham, Mass.):

| | | |
|---|---|---|
| Step 1 | 45° C. | 10 min |
| Step 2 | 45° C. | −0.3° C./cycle, 2 seconds/cycle |

| | |
|---|---|
| Step 3 | go to 2 for 33 cycles |

| | | |
|---|---|---|
| Step 4 | 35° C. | 5 min |
| Step 5 | 45° C. | 5 min |
| Step 6 | 45° C. | 0.2° C./cycle, 1 sec/cycle |

| | |
|---|---|
| Step 7 | go to 7 for 49 cycles |

| | | |
|---|---|---|
| Step 8 | 55° C. | 0.1° C./cycle, 12 sec/cycle |

| | |
|---|---|
| Step 9 | go to 8 for 49 cycles |

| | | |
|---|---|---|
| Step 10 | 55° C. | 2 min |
| Step 11 | 60° C. | 2 min |

| | |
|---|---|
| Step 12 | go to 11 for 9 times |

| | |
|---|---|
| Step 13 | 4° C. forever |
| Step 14 | end |

B. First Strand cDNA Synthesis Method 2

| | |
|---|---|
| mRNA (10 μg) | 25 μL |
| water | 30 μL |
| *Not I adapter primer (5 μg) | 10 μL |
| 65° C. for 10 min, chill on ice, | |
| then add the following reagents, | |
| *5x first buffer | 20 μL |
| *0.1M DTT | 10 μL |
| *10 mM dNTP mix | 5 μL |

Incubate at 45° C. for 2 min, then add 10 μL of *Superscript II RT (200 u/μL), start the following program:

| | |
|---|---|
| Step 1 | 45° C. for 6 sec, −0.1° C./cycle |
| Step 2 | go to 1 for 99 additional cycles |
| Step 3 | 35° C. for 5 min |
| Step 4 | 45° C. for 60 min |
| Step 5 | 50° C. for 10 min |
| Step 6 | 4° C. forever |
| Step 7 | end |

After the 1^{st} strand cDNA synthesis, the DNA is extracted by phenol according to standard procedures, and then precipitated in NaOAc and ethanol, and stored in −20° C.

C. Oxidization of the Diol Group of mRNA for Biotin Labeling

First strand cDNA is spun down and washed once with 70% EtOH. The pellet resuspended in 23.2 μL of DEPC treated water and put on ice. Prepare 100 mM of NaIO4 freshly, and then add the following reagents:

| | |
|---|---|
| mRNA: 1^{st} cDNA (start with 20 μg mRNA) | 46.4 μL |
| 100 mM NaIO4 (freshly made) | 2.5 μL |
| NaOAc 3M pH4.5 | 1.1 μL |

To make 100 mM NaIO4, use 21.39 μg of NaIO4 for 1 μL of water. Wrap the tube in a foil and incubate on ice for 45 min. After the incubation, the reaction is then precipitated in:

| | |
|---|---|
| 5M NaCl | 10 μL |
| 20% SDS | 0.5 μL |
| isopropanol | 61 μL |

Incubate on ice for at least 30 min, then spin it down at max speed at 4° C. for 30 min and wash once with 70% ethanol and then 80% EtOH.

D. Biotinylation of the mRNA Diol Group

Resuspend the DNA in 110 μL DEPC treated water, then add the following reagents:

| | |
|---|---|
| 20% SDS | 5 μL |
| 2M NaOAc pH 6.1 | 5 μL |
| 10 mm biotin hydrazide (freshly made) | 300 μL |

Wrap in a foil and incubate at room temperature overnight.

E. RNase I Treatment

Precipitate DNA in:

| | | |
|---|---|---|
| 5M NaCl | 10 | μL |
| 2M NaOAc pH 6.1 | 75 | μL |
| biotinylated mRNA:cDNA | 420 | μL |
| 100% EtOH (2.5 Vol) | 1262.5 | μL |

(Perform this precipitation in two tubes and split the 420 μL of DNA into 210 μL each, add 5 μL of 5M NaCl, 37.5 μL of 2M NaOAc pH 6.1, and 631.25 μL of 100% EtOH). Store at −20° C. for at least 30 min. Spin the DNA down at 4° C. at maximal speed for 30 min. and wash with 80% EtOH twice, then dissolve DNA in 70 μL RNase free water. Pool two tubes and end up with 140 μL.

Add the following reagents:

| | |
|---|---|
| RNase One 10 U/μL | 40 μL |
| 1^{st} cDNA:RNA | 140 μL |
| 10× buffer | 20 μL |

Incubate at 37° C. for 15 min.

Add 5 μL of 40 μg/μL yeast tRNA to each sample for capturing.

F. Full Length 1^{st} cDNA Capturing Blocking the beads with yeast tRNA:

| | |
|---|---|
| Beads | 1 ml |
| Yeast tRNA 40 μg/μL | 5 μL |

Incubate on ice for 30 min with mixing, wash 3 times with 1 ml of 2M NaCl, 50 mmEDTA, pH 8.0.

Resuspend the beads in 800 μL of 2M NaCl, 50 mm EDTA, pH 8.0, add RNase I treated sample 200 μL, and incubate the reaction for 30 min at room temperature. Capture the beads using the magnetic stand, save the supernatant, and start following washes:
2 washes with 2M NaCl, 50 mm EDTA, pH 8.0, 1 ml each time,
1 wash with 0.4% SDS, 50 μg/ml tRNA,
1 wash with 10 mm Tris-Cl pH 7.5, 0.2 mm EDTA, 10 mm NaCl, 20% glycerol,
1 wash with 50 μg/ml tRNA,
1 wash with 1^{st} cDNA buffer
   G. Second Strand cDNA Synthesis

Resuspend the Beads in:

| | | |
|---|---|---|
| *5× first buffer | 8 | μL |
| *0.1 mM DTT | 4 | μL |
| *10 mM dNTP mix | 8 | μL |
| *5× 2nd buffer | 60 | μL |
| *E. coli Ligase 10 U/μL | 2 | μL |
| *E. coli DNA polymerase 10 U/μL | 8 | μL |
| *E. coli RNaseH 2 U/μL | 2 | μL |
| P32 dCTP 10 μci/μL | 2 | μL |
| Or water up to 300 μL | 208 | μL |

Incubate at 16° C. for 2 hr with mixing the reaction in every 30 min.

Add 4 μL of T4 DNA polymerase and incubate for additional 5 min at 16° C.

Elute 2^{nd} cDNA from the beads.

Use a magnetic stand to separate the 2 d cDNA from the beads, then resuspend the beads in 200 μL of water, and then separate again, pool the samples (about 500 μL). Add 200 μL of water to the beads, then 200 μL of phenol:chloroform, vortex, and spin to separate the sample with phenol.

Pool the DNA together (about 700 μL) and use phenol to clean the DNA again, DNA is then precipitated in 2 μg of glycogen and 0.5 vol of 7.5M NH4OAc and 2 vol of 100% EtOH. Precipitate overnight. Spin down the pellet and wash with 70% EtOH, air-dry the pellet.

| | | | |
|---|---|---|---|
| DNA | 250 μL | DNA | 200 μL |
| 7.5M NH4OAc | 125 μL | 7.5M NH4OAc | 100 μL |
| 100% EtOH | 750 μL | 100% EtOH | 600 μL |
| glycogen 1 μg/μl | 2 μL | glycogen 1 μg/μl | 2 μL |

H. Sal I Adapter Ligation

Resuspend the pellet in 26 μL of water and use 1 μL for TAE gel.

Set up reaction as following:

| | |
|---|---|
| 2^{nd} strand cDNA | 25 μL |
| *5× T4 DNA ligase buffer | 10 μL |
| *Sal I adapters | 10 μL |
| *T4 DNA ligase | 5 μL |

Mix gently, incubate the reaction at 16° C. overnight.
Add 2 μL of ligase second day and incubate at room temperature for 2 hrs (optional).

Add 50 μL water to the reaction and use 100 μL of phenol to clean the DNA, 90 μL of the upper phase is transferred into a new tube and precipitate in:

| | |
|---|---|
| Glycogen 1 μg/μL | 2 μL |
| Upper phase DNA | 90 μL |
| 7.5M NH4OAc | 50 μL |
| 100% EtOH | 300 μL |

precipitate at −20° C. overnight
Spin down the pellet at 4° C. and wash in 70% EtOH, dry the pellet.

I. Not I digestion

| | |
|---|---|
| 2^{nd} cDNA | 41 μL |
| *Reaction 3 buffer | 5 μL |
| *Not I 15 u/μL | 4 μL |

Mix gently and incubate the reaction at 37° C. for 2 hr.
Add 50 μL of water and 100 μL of phenol, vortex, and take 90 μL of the upper phase to a new tube, then add 50 μL of NH4OAc and 300 μL of EtOH.
Precipitate overnight at −20° C.

Cloning, ligation, and transformation are performed per the Superscript cDNA synthesis kit.

### EXAMPLE 3

### Description of cDNA Sequencing and Libraby Subtraction

Individual colonies can be picked and DNA prepared either by PCR with M13 forward primers and M13 reverse primers, or by plasmid isolation. cDNA clones can be sequenced using M13 reverse primers.

cDNA libraries are plated out on 22×22 cm² agar plate at a density of about 3,000 colonies per plate. The plates are incubated in a 37° C. incubator for 12-24 hours. Colonies are picked into 384-well plates by a robot colony picker, Q-bot (GENETIX Limited). These plates are incubated overnight at 37° C. Once sufficient colonies are picked, they are pinned onto 22×22 cm² nylon membranes using Q-bot. Each membrane holds 9,216 or 36,864 colonies. These membranes are placed onto an agar plate with an appropriate antibiotic. The plates are incubated at 37° C. overnight.

After colonies are recovered on the second day, these filters are placed on filter paper prewetted with denaturing solution for four minutes, then incubated on top of a boiling water bath for an additional four minutes. The filters are then placed on filter paper prewetted with neutralizing solution for four minutes. After excess solution is removed by placing the filters on dry filter papers for one minute, the colony side of the filters is placed into Proteinase K solution, incubated at 37° C. for 40-50 minutes. The filters are placed on dry filter papers to dry overnight. DNA is then cross-linked to nylon membrane by UV light treatment.

Colony hybridization is conducted as described by Sambrook, J., Fritsch, E. F. and Maniatis, T., (in Molecular Cloning: A laboratory Manual, 2^{nd} Edition). The following probes can be used in colony hybridization:
1. First strand cDNA from the same tissue as the library was made from to remove the most redundant clones.
2. 48-192 most redundant cDNA clones from the same library based on previous sequencing data.
3. 192 most redundant cDNA clones in the entire maize sequence database.
4. A Sal-A20 oligo nucleotide: TCG ACC CAC GCG TCC GAA AAA AAA AAA AAA AAA AAA, removes clones containing a poly A tail but no cDNA.
5. cDNA clones derived from rRNA.

The image of the autoradiography is scanned into a computer and the signal intensity and cold colony addresses of each colony is analyzed. Re-arraying of cold-colonies from 384 well plates to 96 well plates is conducted using Q-bot.

### EXAMPLE 4

### The Identification of the Gene from a Computer Homology Search

Gene identities can be determined by conducting BLAST (Basic Local Alignment Search Tool; Altschul, S. F., et al., (1993) J. Mol. Biol. 215:403-410; see also www.ncbi.nlm.nih.gov/BLAST/) searches under default parameters for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences are analyzed for similarity to all publicly available DNA sequences contained in the “nr” database using the BLASTN algorithm. The DNA sequences are translated in all reading frames and compared for similarity to all publicly available protein sequences contained in the “nr” database using the BLASTX algorithm (Gish, W. and States, D. J. Nature Genetics 3:266-272 (1993)) provided by the NCBI. In some cases, the sequencing data from two or more clones containing overlapping segments of DNA are used to construct contiguous DNA sequences.

Sequence alignments and percent identity calculations can be performed using the Megalign program of the LASERGENE bioinformatics computing suite (DNASTAR Inc., Madison, Wis.). Multiple alignment of the sequences can be performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method are KTUPLE 1, GAP PENALTY=3, WINDOW=5 and DIAGONALS SAVED=5.

### EXAMPLE 5

### The Expression of Transgenes in Monocot Cells

A transgene comprising a cDNA encoding the instant polypeptides in sense orientation with respect to the maize 27 kD zein promoter that is located 5′ to the cDNA fragment, and the 10 kD zein 3′ end that is located 3′ to the cDNA fragment, can be constructed. The cDNA fragment of this gene may be generated by polymerase chain reaction (PCR) of the cDNA clone using appropriate oligonucleotide primers. Cloning sites (NcoI or SmaI) can be incorporated into the oligonucleotides to provide proper orientation of the DNA fragment when inserted into the digested vector pML103 as described below. Amplification is then performed in a standard PCR. The amplified DNA is then digested with restriction enzymes NcoI and SmaI and fractionated on an agarose gel. The appropriate band can be isolated from the gel and combined with a 4.9 kb NcoI-SmaI fragment of the plasmid pML103. Plasmid pML 103 has been deposited under the terms of the Budapest Treaty at ATCC (American Type Culture Collection, 10801 University Blvd., Manassas, Va. 20110-2209), and bears accession number ATCC 97366. The DNA segment from pML103 contains a 1.05 kb SalI-NcoI promoter fragment of the maize 27 kD zein gene and a 0.96 kb SmaI-SalI fragment from the 3′ end of the maize 10 kD zein gene in the vector pGem9Zf(+) (Promega). Vector and insert DNA can be ligated at 15° C. overnight, essentially as described (Maniatis). The ligated DNA may then be used to transform E. coli XL 1-Blue (Epicurian Coli XL-1 Blue; Stratagene). Bacterial transformants can be screened by restriction enzyme digestion of plasmid DNA and limited nucleotide sequence analysis using the dideoxy chain termination method (Sequenase DNA Sequencing Kit; U.S. Biochemical). The resulting plasmid construct would comprise a transgene encoding, in the 5′ to 3′ direction, the maize 27 kD zein promoter, a cDNA fragment encoding the instant polypeptides, and the 10 kD zein 3′ region.

The transgene described above can then be introduced into corn cells by the following procedure. Immature corn embryos can be dissected from developing caryopses derived from crosses of the inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al. (1975) Sci. Sin. Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The plasmid, p35S/Ac (Hoechst Ag, Frankfurt, Germany) or equivalent may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35 S/Ac is under the control of the ³⁵S promoter from Cauliflower Mosaic Virus (Odell et al. (1985) Nature 313:810-812) and the 3′ region of the nopaline synthase gene from the T-DNA of the Ti plasmid of Agrobacterium tumefaciens.

The particle bombardment method (Klein et al. (1987) Nature 327:70-73) may be used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton flying disc (Bio-Rad Labs). The particles are then accelerated into the corn tissue with a Biolistic PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains gluphosinate (2 mg per liter) and lacks casein or proline. The tissue continues to grow slowly on this medium. After an additional 2 weeks the tissue can be transferred to fresh N6 medium containing gluphosinate. After 6 weeks, areas of about 1 cm in diameter of actively growing callus can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al. (1990) Bio/Technology 8:833-839).

### EXAMPLE 6

### The Expression of Transgenes in Dicot Cells

A seed-specific expression cassette composed of the promoter and transcription terminator from the gene encoding the β subunit of the seed storage protein phaseolin from the bean Phaseolus vulgaris (Doyle et al. (1986) J. Biol. Chem. 261:9228-9238) can be used for expression of the instant polypeptides in transformed soybean. The phaseolin cassette includes about 500 nucleotides upstream (5′) from the translation initiation codon and about 1650 nucleotides downstream (3′) from the translation stop codon of phaseolin. Between the 5′ and 3′ regions are the unique restriction endonuclease sites Nco I (which includes the ATG translation initiation codon), SmaI, KpnI and XbaI. The entire cassette is flanked by Hind III sites.

The cDNA fragment of this gene may be generated by polymerase chain reaction (PCR) of the cDNA clone using appropriate oligonucleotide primers. Cloning sites can be incorporated into the oligonucleotides to provide proper orientation of the DNA fragment when inserted into the expression vector. Amplification is then performed as described above, and the isolated fragment is inserted into a pUC18 vector carrying the seed expression cassette.

Soybean embroys may then be transformed with the expression vector comprising sequences encoding the instant polypeptides. To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos which produce secondary embryos are then excised and placed into a suitable liquid medium. After repeated selection for clusters of somatic embryos which multiplied as early, globular staged embryos, the suspensions are maintained as described below. Soybean embryogenic suspension cultures can be maintained in 35 mL liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium.

Soybean embryogenic suspension cultures may then be transformed by the method of particle gun bombardment (Klein et al. (1987) Nature (London) 327:70-73, U.S. Pat. No. 4,945,050). A Du Pont Biolistic PDS1000/HE instrument (helium retrofit) can be used for these transformations.

A selectable marker gene which can be used to facilitate soybean transformation is a transgene composed of the ³⁵S promoter from Cauliflower Mosaic Virus (Odell et al. (1985) Nature 313:810-812), the hygromycin phosphotransferase gene from plasmid pJR225 (from E. coli; Gritz et al.(1983) Gene 25:179-188) and the 3′ region of the nopaline synthase gene from the T-DNA of the Ti plasmid of Agrobacterium tumefaciens. The seed expression cassette comprising the phaseolin 5′ region, the fragment encoding the instant polypeptide and the phaseolin 3′ region can be isolated as a restriction fragment. This fragment can then be inserted into a unique restriction site of the vector carrying the marker gene.

To 50 μL of a 60 mg/mL 1 μm gold particle suspension is added (in order): 5 μL DNA (1 μg/μL), 20 μL spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches of mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### EXAMPLE 7

### The Expression of a Transgene in Microbial Cells

The cDNAs encoding the instant polypeptides can be inserted into the T7 E. coli expression vector pBT430. This vector is a derivative of pET-3a (Rosenberg et al. (1987) Gene 56:125-135) which employs the bacteriophage T7 RNA polymerase/T7 promoter system. Plasmid pBT430 was constructed by first destroying the EcoR I and Hind III sites in pET-3a at their original positions. An oligonucleotide adaptor containing EcoR I and Hind III sites was inserted at the BamH I site of pET-3a. This created pET-3aM with additional unique cloning sites for insertion of genes into the expression vector. Then, the Nde I site at the position of translation initiation was converted to an Nco I site using oligonucleotide-directed mutagenesis. The DNA sequence of pET-3aM in this region, 5′-CATATGG, was converted to 5′-CCCATGG in pBT430.

Plasmid DNA containing a cDNA may be appropriately digested to release a nucleic acid fragment encoding the protein. This fragment may then be purified on a 1% NuSieve GTG low melting agarose gel (FMC). Buffer and agarose contain 10 μg/mL ethidium bromide for visualization of the DNA fragment. The fragment can then be purified from the agarose gel by digestion with GELase (Epicentre Technologies) according to the manufacturer's instructions, ethanol precipitated, dried and resuspended in 20 μL of water. Appropriate oligonucleotide adapters may be ligated to the fragment using T4 DNA ligase (New England Biolabs, Beverly, Mass.). The fragment containing the ligated adapters can be purified from the excess adapters using low melting agarose as described above. The vector pBT430 is digested, dephosphorylated with alkaline phosphatase (NEB) and deproteinized with phenol/chloroform as described above. The prepared vector pBT430 and fragment can then be ligated at 16° C. for 15 hours followed by transformation into DH5 electrocompetent cells (GIBCO BRL). Transformants can be selected on agar plates containing LB media and 100 μg/mL ampicillin. Transformants containing the gene encoding the instant polypeptides are then screened for the correct orientation with respect to the T7 promoter by restriction enzyme analysis.

For high level expression, a plasmid clone with the cDNA insert in the correct orientation relative to the T7 promoter can be transformed into E. coli strain BL21(DE3) (Studier et al. (1986) J. Mol. Biol. 189:113-130). Cultures are grown in LB medium containing ampicillin (100 mg/L) at 25° C. At an optical density at 600 nm of approximately 1, IPTG (isopropylthio-p-galactoside, the inducer) can be added to a final concentration of 0.4 mM and incubation can be continued for 3 h at 25°. Cells are then harvested by centrifugation and re-suspended in 50 μL of 50 mM Tris-HCl at pH 8.0 containing 0.1 mM DTT and 0.2 mM phenyl methylsulfonyl fluoride. A small amount of 1 mm glass beads can be added and the mixture sonicated 3 times for about 5 seconds each time with a microprobe sonicator. The mixture is centrifuged and the protein concentration of the supernatant determined. One microgram of protein from the soluble fraction of the culture can be separated by SDS-polyacrylamide gel electrophoresis. Gels can be observed for protein bands migrating at the expected molecular weight.

The above examples are provided to illustrate the invention but not to limit its scope. Other variants of the invention will be readily apparent to one of ordinary skill in the art and are encompassed by the appended claims. All publications, patents, patent applications, and computer programs cited herein are hereby incorporated by reference.

1. An isolated nucleic acid encoding a polypeptide selected from the group consisting of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, and 208.

2-25. (canceled)

26. An isolated polynucleotide comprising:
(a) a nucleotide sequence encoding a polypeptide having anthranilate N-hydroxycinnamoyl/benzoyltransferases activity, wherein the amino acid sequence of the polypeptide and the amino acid sequence of SEQ ID NO:52 have at least 80% sequence identity based on the Clustal alignment method with default pairwise alignment parameters of KTUPLE=1, GAP PENALTY=3, WINDOW=5 and DIAGONALS SAVED=5, or
(b) the complement of the nucleotide sequence of (a).

27. The polynucleotide of claim 26, wherein the amino acid sequence of the polypeptide and the amino acid sequence of SEQ ID NO:52 have at least 85% sequence identity based on the Clustal alignment method with the default pairwise alignment parameters.

28. The polynucleotide of claim 26, wherein the amino acid sequence of the polypeptide and the amino acid sequence of SEQ ID NO:52 have at least 90% sequence identity based on the Clustal alignment method with the default pairwise alignment parameters.

29. The polynucleotide of claim 26, wherein the amino acid sequence of the polypeptide and the amino acid sequence of SEQ ID NO:52 have at least 95% sequence identity based on the Clustal alignment method with the default pairwise alignment parameters.

30. The polynucleotide of claim 26, wherein the nucleotide sequence comprises the nucleotide sequence of SEQ ID NO:51.

31. The polynucleotide of claim 26, wherein the amino acid sequence of the polypeptide comprises the amino acid sequence of SEQ ID NO:52.

32. A vector comprising the polynucleotide of claim 26.

33. A recombinant DNA construct comprising the polynucleotide of claim 26 operably linked to at least one regulatory sequence.

34. A method for transforming a cell comprising transforming a cell with the polynucleotide of claim 26.

35. A cell comprising the recombinant DNA construct of claim 33.

36. A method for producing a plant comprising transforming a plant cell with the polynucleotide of claim 26 and regenerating a plant from the transformed plant cell.

37. A plant comprising the recombinant DNA construct of claim 33.

38. A seed comprising the recombinant DNA construct of claim 33.
